SEQUENCE LISTING (108 sequences)

SEQ ID NO: 1 - DNA, 39 bp, Campylobacter coli
tttgtgaaatctaatggcttaaccattaaactgcttgag

SEQ ID NO: 2 - DNA, 29 bp, Campylobacter coli
atccgtagagatcaccaagaatacccatt

SEQ ID NO: 3 - DNA, 54 bp, Campylobacter jejuni
gtctcttgtgaaatctaatggcttaaccattaaactgcttgggaaactgatagt

SEQ ID NO: 4 - DNA, 24 bp, Campylobacter jejuni
ggaactcaactgacgctaaggcgc

SEQ ID NO: 5 - DNA, 24 bp, Pseudomonas aeruginosa
gtggttcagcaagttggatgtgaa

SEQ ID NO: 6 - DNA, 27 bp, Pseudomonas aeruginosa
aaactactgagctagagtacggtagag

SEQ ID NO: 7 - DNA, 35 bp, Pseudomonas fluorescens
tgttttgacgttaccgacagaataagcaccggcta

SEQ ID NO: 8 - DNA, 27 bp, Pseudomonas fluorescens
tagagtatggtagagggtggtggaatt

SEQ ID NO: 9 - DNA, 21 bp, Legionella
atactgacactgaggcacgaa

SEQ ID NO: 10 - DNA, 23 bp, Legionella pneumophila
ttactgggcgtcaagggtgcgta

SEQ ID NO: 11 - DNA, 23 bp, Legionella pneumophila
ttaacctgggacggtcagataat

SEQ ID NO: 12 - DNA, 27 bp, Acinetobacter baumannii
gcgtaggcggcttattaagtcggatgt

SEQ ID NO: 13 - DNA, 27 bp, Acinetobacter baumannii
cattcgatactggtgagctagagtatg

SEQ ID NO: 14 - DNA, 47 bp, Escherichia Coli Shigella Species
cggggaggaagggagtaaagttaatacctttgctcattgacgttacc

SEQ ID NO: 15 - DNA, 22 bp, Salmonella
gaggaaggtgttgtggttaata

SEQ ID NO: 16 - DNA, 32 bp, Aeromonas caviae
cagtagctaatatctgctggctgtgacgttac

SEQ ID NO: 17 - DNA, 32 bp, Aeromonas hydrophila
acgcaggcggttggataagttagatgtgaaag

SEQ ID NO: 18 - DNA, 20 bp, Aeromonas hydrophila
aattgcatttaaaactgtcc

SEQ ID NO: 19 - DNA, 35 bp, Aeromonas sobria
gaaaggttggcagctaatatctgtcagctgtgacg

SEQ ID NO: 20 - DNA, 26 bp, Aeromonas sobria
aattgctgttcagctagagtcttgta

SEQ ID NO: 21 - DNA, 53 bp, Vibrio cholerae
cagtagggaggaaggtggttaagttaataccttaatcatttgacgttacctac

SEQ ID NO: 22 - DNA, 48 bp, Vibrio cholerae
tcaacctaggaatcgcatttgaaactgacaagctagagtactgtagag

SEQ ID NO: 23 - DNA, 37 bp, Staphylococcus aureus
gttattagggaagaacatatgtgtaagtaactgtgca

SEQ ID NO: 24 - DNA, 37 bp, Staphylococcus epidermidis
tattagggaagaacaaatgtgtaagtaactatgcacg

SEQ ID NO: 25 - DNA, 23 bp, Streptococcus bovis Streptococcus equinus
ttggaaactgttagacttgagtg

SEQ ID NO: 26 - DNA, 43 bp, Enterococcus faecalis
aagaacaaggacgttagtaactgaacgtcccctgacggtatct

SEQ ID NO: 27 - DNA, 27 bp, Enterococcus faecalis, hirae, durans
agagtaactgttcatcccttgacggta

SEQ ID NO: 28 - DNA, 29 bp, Clostridium perfringens
agcgtaggcggatgattaagtgggatgtg

SEQ ID NO: 29 - DNA, 22 bp, Clostridium perfringens
gtgctgcattccaaactggtta

SEQ ID NO: 30 - DNA, 24 bp, Mycobacterium sp.
gcgtgcgggcgatacgggcagact

SEQ ID NO: 31 - DNA, 25 bp, Mycobacterium avium, intracellulare
aaggtccgggttttctcggattgac

SEQ ID NO: 32 - DNA, 29 bp, Mycobacterium kansasii
gtccgggttctctcggattgacggtaggt

SEQ ID NO: 33 - DNA, 22 bp, Mycobacterium gordonae
gttttctcgggctgacggtagg

SEQ ID NO: 34 - DNA, 24 bp, Mycobacterium marinum
aggttcgggttttctcggattgac

SEQ ID NO: 35 - DNA, 20 bp, Mycobacterium xenopi
ctttcagcctcgacgaagct

SEQ ID NO: 36 - DNA, 22 bp, Mycobacterium xenopi
gtgacggtaggggcagaagaag

SEQ ID NO: 37 - DNA, 42 bp, Burkholderia gladioli
ccggaaagaaatcctgagggctaatatccttcggggatgacg

SEQ ID NO: 38 - DNA, 25 bp, Burkholderia cepacia
actgcattggtgactggcaggctag

SEQ ID NO: 39 - DNA, 39 bp, Stenotrophomonas maltophilia
gaggaacatccatggcgaaggcagctacctggaccaaca

SEQ ID NO: 40 - DNA, 23 bp, Cryptosporidium
cagttatagtttacttgataatc

SEQ ID NO: 41 - DNA, 20 bp, Cryptosporidium
ttattagataaagaaccaat

SEQ ID NO: 42 - DNA, 27 bp, Cryptosporidium
acctatcagctttagacggtagggtat

SEQ ID NO: 43 - DNA, 27 bp, Cryptosporidium
tgccttgaatactccagcatggaataa

SEQ ID NO: 44 - DNA, 37 bp, Cryptosporidium
agagattggaggttgttccttactccttcagcacctt

SEQ ID NO: 45 - DNA, 35 bp, Cryptosporidium parvum
tcattataacagttatagtttacttgataatcttt

SEQ ID NO: 46 - DNA, 43 bp, Cryptosporidium parvum
attggaatgagttaagtataaacccctttacaagtatcaattg

SEQ ID NO: 47 - DNA, 29 bp, Cryptosporidium parvum
tagttggatttctgttaataatttatata

SEQ ID NO: 48 - DNA, 36 bp, Cryptosporidium parvum
atatttatataatattaacataattcatattactat

SEQ ID NO: 49 - DNA, 41 bp, Cryptosporidium parvum
tttcgaaggaaatgggtaatcttttgaatatgcatcgtgat

SEQ ID NO: 50 - DNA, 382 bp, Adenovirus 40 (L19443)
ctaaagggaactgccagtgtaaagcataacatgatttgtggcggtggtcactctcagctgctaacctgtgcagatggaaactgtcaggctctgagagtgtttcacgtagtatctcatccccgccgcccctggcctgtttttgagcacaacatgcttatgcgctgtactgtgcatttgggagctcgtcgtggcatgttttctccataccagagtaacttttgccacactaaagttttaatggaaactgatgctttttctcgggtatggtggaacggggtatttgatttaaccatggagctatttaaagtggtgaggtatgatgagtcaaaggttcgttgtcgcccctgtgagtgtggagctaatcatattaggttatatccag

SEQ ID NO: 51 - DNA, 382 bp, Adenovirus 41 bis (M18289)
ctgaagggaactgccagtgttaagcataatatgatttgtggcactggtcactctcagctgctaacttgcgcagatggaaactgtcagactctaaaagtgattcatgtggtttcccatcagcgccgcccctggcctgtttttgagcataacatgcttatgcgttgtaccatgcatttgggggctcgtcgtggcatgttttctccatatcagagtaatttttgccatactaaagttttaatggaaactgatgctttttcgcgggtgtggtggagcggggtgtttgatttgaccatagagctgtataaagtggtgagatatgatgagttaaaggctcgttgtcgcccctgtgagtgtggagccaatcacatcaggttatatccag

SEQ ID NO: 52 - DNA, 67 bp, Astrovirus HAstV-1-2 (L23513)
agggtacagcttccttcttttctgtctctgtttagattattttaatcaccatttaaaattgatttaa

SEQ ID NO: 53 - DNA, 521 bp, Poliovirus (X00595)
cggtacctttgtgcgcctgttttatactcccctcccgcaacttagaagcacgaaaccaagttcaatagaagggggtacaaaccagtaccactacgaacaagcacttctgtttccccggtgacattgcatagactgctcacgcggttgaaagtgatcgatccgttacccgcttgtgtacttcgaaaagcctagtatcgccttggaatcttcgacgcgttgcgctcagcacccgaccccggggtgtggcttaggctgatgagtctggacattcctcaccggtgacggtggtctaggctgcgttggcggcctacctatggctaacgccataggacgttagatgtgaacaaggtgtgaagagcctattgagctacataagagtcctccggcccctgaatgcggctaatcctaaccacggaacaggcggtcgcgaaccagtgactggcttgtcgtaacgcgcaagtctgtggcggaaccgactactttgggtgtccgtgtttcctgttatttttatcatggctgct

SEQ ID NO: 54 - DNA, 520 bp, Coxsackievirus (D00538)
aggtacctttgtacgcctgttttatatcccttcccccgtaactttagaagcttatcaaaagttcaatagcaggggtacaagccagtacctctacgaacaagcacttctgtttccccggtgaaatcatatagactgtacccacggtcaaaagtgattgatccgttatccgcttgagtacttcgagaagcctagtatcgccttggaatcttcgacgcgttgcgctcaacactctgccccgagtgtagcttaggctgatgagtctgggcactccccaccggcgacggtggcccaggctgcgttggcggcctacccatggctgatgccgtgggacgctagttgtgaacaaggtgtgaagagcctattgagctactcaagagtcctccggcccctgaatgcggctaatcctaaccacggagcaatcgctcacgacccagtgagtaggttgtcgtaatgcgtaagtctgtggcggaaccgactactttgggtgtccgtgtttccctttatattcatactggctgct

SEQ ID NO: 55 - DNA, 525 bp, Echovirus (X77708)
cggtacctttgtgcgcctgttttatataccctcccctcagtaacctagaagttcatcacaaatgatcaatagttagctcaacaaaccagttgagcctagatcaagcacttctgttaccccgggctgagtatcaataagctgttgacacggctgaaggagaaaacgcccgttacccgaccagctacttcggagaacctagtatcaccatagaggttgcgtagcgtttcgctccgcacaaccccagtgtagatcaggtcgatgagtcaccgcgttccccacaggcgactgtggcggtggctgcgttggcggcctgcccatggggttacccatgggacgcttcaatactgacatggtgtgaagagttgactgagctagctggtagtcctccggcccctgaatgcggctaatcctaactgtggagcaagtgcccacaacccagtgggtggcttgtcgtaatgggcaactctgcagcggaaccgactactttgggtgaccgtgtttctctttattcttatattggctgct

SEQ ID NO: 56 - DNA, 981 bp, Rotavirus U36242
atgtatggtattgaatataccacaattctgaccattttgatatttatcatattattgaattatatattaaaaactataactaatacgatggactatatagtttttaaatttttgctactaatcgctctgatgtcaccatttgtaaggacgcaaaattatggcatgtatttaccaataacaggatcactagacgctgtatacacaaattcaactagtggagaatcatttctaacttcaacgctatgtttatactatccaacagaagctaaaaatgagatttcagataatgaatgggaaaatactctatcagaattatttttaactaaaggatggccggctggatcagtttattttaaagactacaatgatattactacattttctatgaatccacaactgtattgtgattataatgtagtattgatgagatatgataatacatctgaattagatgcatcggagttagcagatcttatattgaacgaatggctgtgcaatcctatggatatatcactttactattatcaacaaaatagcgaatcaaacaaatggatatcaatcggaacagactgtacggtaaaagtttgtccactcaatacacaaactctaggaattggatgcaaaactacggacgtggatacatttgagattgttgcgtcgtctgaaaaattggtaattactgatgttgtaaatggtgtaaaccataaaataaatatttcaataagtacatgtactatacgtaattgtaataaactaggaccacgagaaaatgttgctataattcaagttggtggaccgaacgcactagatatcactgctgatccaacaacagttccacaggttcaaagaattatgcgagtaaattggaaaaaatggtggcaagtgttttatacagtagttgactatattaaccaaattatacaagttatgtccaaacggtcaagatcattagacacagctgctttttattatagaatttag

SEQ ID NO: 57 - DNA, 981 bp, Rotavirus M86834
atgtatggtattgaatataccacagttctattttatttgatatcgttcgttcttgtgagttacattttaaaaaccataacgaaaatgatggactatattatttatagagtaacttttataattgttgtattatcagtactgtctaatgcgcaaaattatggaataaatttgccaattactggatctatggatacagcgtacgctaattcgacgcaaaatggaaatttcctgtcttcaactctatgtctatattatccatctgaggctccaactcaaattagtgataacgaatggaaagatacattatctcagttgtttttgactaagggatggccaacaggttcagtttattttaatgaatattcgaatgttctggatttttcaattgacccaaaattatactgtgattataatattgtattaattaaatttgcttctggagaggagttggatatatctgaactagctgatctgatactaaatgaatggttgtgtaatccaatggatataacgctatatgattatcaacaaactggagaagcaaataaatggatatcaatgggatcatcttgtactgtcaaagtgtgcccattaaatacgcaaactttaggaattggctgccaaacaacgaatgtagctacttttgaaatggtggctgacagtgaaaaactagcgatagttgatgttgttgataatgtaaatcataaattagatattacatctacaacgtgtacaatacgaaattgtaagaaattaggtccaagagaaaatgtggctataatacaggttggtggttctaatatactagatataacggctgatcccacgacttcaccgcaaacggaacgaatgatgcgtgttaattggaagaaatggtggcaagtattttacactgtagttgattatattaatcagatagtacaaatgatgtccaaaagatcgaggtcgctagattcatcctctttttattatagagtatag

SEQ ID NO: 58 - DNA, 981 bp, Rotavirus U26395
atgtatggtattgaatataccacaattctaatctttctgatatcaatcatcctactcaactatatattaaaatcagtgacccgaataatggactacattatatatagatttttattaatttctgtagcattatttgccttaactaaagctcagaactatggacttaatataccaataacaggatcaatggacactgtttactccaactctactcaagaaggaatatttctaacatccacattatgtttgtattatccaactgaagcaagtactcaaatcagtgatggtgattggaaagactcattatcacaaatgtttcttacaaaaggttggccaacaggatcagtctattttaaagagtactcaaatattgttgacttttccgttgatccacaattatattgtgattataacttagtactaatgaagtatgatcaaaatcttgaactagatatgtcagaattagctgatttgatattgaatgaatggctatgtaatccaatggatataacattatattattatcaacaatcgggagaatcaaataagtggatatcaatgggatcatcatgtactgtgaaagtgtgtccactgaatacacaaacgttaggaataggttgtcaaacaacgaatgtagactcatttgaaacggttgctgaaaatgaaaaattagctatagtggatgtcgttgatgggatcaatcataaaataaatttgacaactacgacatgtactattcgaaattgtaagaagttaggtccaagagagtatgtagctatcatacaagttggtggctctaatatattagacataacagcggatccagcgactaatccacaaattgagagaatgatgagagtgaattggaaaagatggtggcaagtattttataccatagtagattatattaatcagattgtacaggtgatgtccaaaagatcaagatcattaaattctgcagctttttattatagagtatag

SEQ ID NO: 59 - DNA, 398 bp, Virus de Norwalk (M87661)
ataaaagttggcatgaacacaatagaagatggccccctcatctatgctgagcatgctaaatataagaatcattttgatgcagattatacagcatgggactcaacacaaaatagacaaattatgacagaatccttctccattatgtcgcgccttacggcctcaccagaattggccgaggttgtggcccaagatttgctagcaccatctgagatggatgtaggtgattatgtcatcagggtcaaagaggggctgccatctggattcccatgtacttcccaggtgaacagcataaatcactggataattactctctgtgcactgtctgaggccactggtttatcacctgatgtggtgcaatccatgtcatatttctcattttatggtgatgatgagattgt

SEQ ID NO: 60 - DNA, 247 bp, Hepatite A (M14707)
gttttgctcctctttatcatgctatggatgttactacacaagttggagatgattctggaggtttttcaacaacagtttctacagaacagaatgttccagatccccaagttggtataacaaccatgaaagatttgaaaggaaaagctaacagagggaaaatggatgtttcaggagtacaagcacctgtgggagctatcacaacaattgaggatccagttttagcaaagaaagtacctgagacatttcc

SEQ ID NO: 61 - DNA, 54 bp, Enterococcus faecalis
tctcaatcactggacgtggtactgttgctacaggacgtgttgaacgtggtgaag

SEQ ID NO: 62 - DNA, 53 bp, Escherichia coli
tctccatctccggtcgtggtaccgttgttaccggtcgtgtagaacgcggtatc

SEQ ID NO: 63 - DNA, 21 bp, Cryptosporidium parvum
tcctacgtctaacttcacgtg

SEQ ID NO: 64 - DNA, 21 bp, Cryptosporidium parvum
tgtgattggtaaaaagtatag

SEQ ID NO: 65 - DNA, 164 bp, Giardia lamblia
ggaatgtcttgtaggcgcccgcccccaccgcgcgccggatgcgtccctgccccttgtacacaccgcccgtcgctcctaccgactgggcgcggcggcgagcgccccggacgcgcgaagggccgcgagcccccgcgcctggaggaaggagaagtcgtaacaaggta

SEQ ID NO: 66 - DNA, 23 bp, Escherichia coli
tacctttgctcattgacgttacc

SEQ ID NO: 67 - DNA, 24 bp, Enterococcus faecalis
actgaacgtcccctgacggtatct

SEQ ID NO: 68 - DNA, 51 bp, Escherichia coli O157 H7
tggatcgcgaaaactgtggaattgagcagcgttggtgggaaagcgcgttac

SEQ ID NO: 69 - DNA, 81 bp, Escherichia coli
tgtgggcattcagtctggatcgcgaaaactgtggaattgagcagcgttggtgggaaagcgcgttacaagaaagccgggcaa

SEQ ID NO: 70 - DNA, 30 bp, Poliovirus type 2
ctccggcccctgaatgcggctaatcctaac

SEQ ID NO: 71 - DNA, 20 bp, Poliovirus type 2
accagtgactggcttgtcgt

SEQ ID NO: 72 - DNA, 30 bp, Coxsackievirus A21
tccggcccctgaatgcggctaatcctaacc

SEQ ID NO: 73 - DNA, 20 bp, Coxsackievirus A21
ccagtgagtaggttgtcgta

SEQ ID NO: 74 - DNA, 30 bp, Echovirus 12
agtcctccggcccctgaatgcggctaatcc

SEQ ID NO: 75 - DNA, 20 bp, Echovirus 12
acaacccagtgggtggcttg

SEQ ID NO: 76 - DNA, 1061 bp, Rotavirus
ggctttaaaagagagaatttccgtttggctagcggttagctccttttaatgtatggtattgaatataccacaattctaacctttctgatatcaatagttttattgaactatatattaaaatcactaactagtgcgatggactttataatttatagatttcttttacttattgttattgcatcaccttttgttaaaacacaaaattatggaattaatttaccgatcactggctccatggatacagcatatgcaaattcatcacagcaagaaacatttttgacttcaacgctatgcttatattatcctacagaagcgtcaactcaaattggagatacagaatggaaggatactctgtcccaattattcttgactaaagggtggccaactggatcagtctattttaaagaatacaccgatatcgcttcattctcaattgatccgcaactttattgggattataatgttgtactgatgaagtatgattcaacgttagagctagatatgtctgaattagctgatttaattctaaatgaatggttatgtaacccaatggatataacattatattattatcagcaaacagatgaagcgaataaatggatatcgatgggacagtcttgtaccataaaagtatgtccattgaatacgcagactttaggaataggttgtattaccacaaatacagcgacatttgaagaggtggctacaagtgaaaaattagtaataaccgatgttgttgatggtgtgaaccataaacttgatgtgactacaaatacctgtacaattaggaattgtaagaagttgggaccaagagaaaatgtagcgattatacaagtcggtggctcagatgtgttagatattacagcggatccaactactgcaccacaaactgaacgtatgatgcgagtaaattggaagaaatggtggcaagttttctatacagtagtagattatattaatcagattgtgcaagttatgtccaaaagatcacggtcattaaattcagcagctttttactatagggtttgatatatcttagattagaattgtatgatgtgacc

SEQ ID NO: 77 - DNA, 30 bp, Rotavirus
tttttaaatttttgctactaatcgctctga

SEQ ID NO: 78 - DNA, 21 bp, Rotavirus
aatacatctgaattagatgca

SEQ ID NO: 79 - DNA, 19 bp, Rotavirus
caaacaaatggatatcaat

SEQ ID NO: 80 - DNA, 45 bp, Rotavirus
ggttcaaagaattatgcgagtaaattggaaaaaatggtggcaagt

SEQ ID NO: 81 - DNA, 20 bp, Rotavirus
ctttttattatagaatttag

SEQ ID NO: 82 - DNA, 30 bp, Rotavirus
tttatagagtaacttttataattgttgtat

SEQ ID NO: 83 - DNA, 21 bp, Rotavirus
tctggagaggagttggatata

SEQ ID NO: 84 - DNA, 19 bp, Rotavirus
caaataaatggatatcaat

SEQ ID NO: 85 - DNA, 45 bp, Rotavirus
aacggaacgaatgatgcgtgttaattggaagaaatggtggcaagt

SEQ ID NO: 86 - DNA, 20 bp, Rotavirus
ctttttattatagagtatag

SEQ ID NO: 87 - DNA, 30 bp, Rotavirus
tatatagatttttattaatttctgtagcat

SEQ ID NO: 88 - DNA, 21 bp, Rotavirus
caaaatcttgaactagatatg

SEQ ID NO: 89 - DNA, 19 bp, Rotavirus
caaataagtggatatcaat

SEQ ID NO: 90 - DNA, 45 bp, Rotavirus
aattgagagaatgatgagagtgaattggaaaagatggtggcaagt

SEQ ID NO: 91 - DNA, 20 bp, Rotavirus
ctttttattatagagtatag

SEQ ID NO: 92 - DNA, 30 bp, Rotavirus
tttatagatttcttttacttattgttattg

SEQ ID NO: 93 - DNA, 21 bp, Rotavirus
tcaacgttagagctagatatg

SEQ ID NO: 94 - DNA, 19 bp, Rotavirus
cgaataaatggatatcgat

SEQ ID NO: 95 - DNA, 45 bp, Rotavirus
aactgaacgtatgatgcgagtaaattggaagaaatggtggcaagt

SEQ ID NO: 96 - DNA, 20 bp, Rotavirus
ctttttactatagggtttga

SEQ ID NO: 97 - DNA, 64 bp, Hepatitis A virus
atggatgtttcaggagtacaagcacctgtgggagctatcacaacaattgaggatccagttttag

SEQ ID NO: 98 - DNA, 551 bp, Sapporo Virus
tgtgatgctgccaccacgcttatagccaccgcggcttttaaggccgtggctaccaggctacaggtggtgacaccaatgacaccagttgctgttggcattaacatggactctgttcagatgcaagtgatgaatgactctttaaaggggggtgttctttactgtttggattattccaaatgggattccacacaaaaccctgcagtgacagcagcctccctggcaatattggagagatttgctgagccccatccaattgtgtcttgtgccattgaggctctttcctcccctgcagagggctatgtcaatgatatcaaatttgtgacacgcggcggcctaccatctgggatgccatttacatctgtcgtcaattctatcaaccatatgatatacgtggcggcagccatcctgcaggcatacgaaagccacaatgtcccatatactggaaacgtcttccaagtggagaccgttcacacgtatggtgatgattgcatgtacagcgtgtgccctgccactgcatcaattttccacactgtgcttgcaaacctaacgtc

SEQ ID NO: 99 - DNA, 382 bp, Southampton Virus
tcaaagttggaatgaattcaattgaggatgggccactgatctatgcagaacattcaaaatataagtaccactttgatgcagattacacagcttgggactcaactcaaaataggcaaatcatgacagagtcattttcaatcatgtgtcggctaactgcatcacccgaactagcttcggtggtggctcaagacttgctcgcaccctcagagatggatgttggcgactacgtcataagagtgaaggaaggcctcccatctggtttcccatgcacatcacaagttaatagtataaaccattggttaataactctgtgcgccctttctgaagtgactggcctgtcgccagacgttatccaatccatgtcatatttctctttctatgg

SEQ ID NO: 100 - DNA, 312 bp, Desert Shield Virus
tttgatgctgattacacggcctgggattccactcaaaacagggaaatcatgatggagtcctttaacatcatgtgtaaactcactgccaacccttccctggctgcggtagtggcacaagacttactttctccttctgaaatggatgttggagattatgtaatcagtgtgaaagacggcctgccatcaggcttcccgtgtacctcacaagtcaacagcataaatcactggattctcaccctgtgtgcattgtcagaagtcaccgggctctccccagatgtgttgcagtcacagtcgtatttttccttctatggg

SEQ ID NO: 101 - DNA, 362 bp, Toronto Virus
aatgaagatggtcccataatatttgagaaacattccagatacagataccactatgatgcagattactcccgctgggactccacgcagcagcgggcagtgctggcagcagcacttgaaatcatggtgaggttctctgctgaaccacagctagcacaaatagtagctgaagacctgctagcaccaagtgtggttgatgtgggtgacttcaagatcaccattaatgaaggcctaccttctggtgtgccttgcacctcacagtggaactccattgcccactggttgcttactctgtgtgccctttctgaagttacaggactaggccccgacatcatacaagctaattctatgtactctttctatgg

SEQ ID NO: 102 - DNA, 364 bp, Snow Mountain Virus
tgaatgaggatggacccataatttttgaaaagcactccaggttctcataccactatgatgcagattactcacgctgggactcaacccaacagagggcagtgctagctgcagccttggaaatcatggtaaaattctcaccagaaccacatttggcccaaattgttgcagaggatctcctagcccccagtgtgatggatgtaggtgatttcaaaataacaattaatgagggactgccctcgggagtaccctgcacatcacagtggaattccatcgcccactggctcctcacactctgtgcactatctgaagtcacaaacctggctcctgacatcatacaagctaactccttgttctctttctatgg

SEQ ID NO: 103 - DNA, 376 bp, Hawaii Virus
tcagagttggtatgaacatgaatgaggatggccccattatctttgagaaacactccaggtataaatatcattaagattattctcgatgggactcaacacagcagagagccgtactagctgcagccctagagatcatggtcaaattctccccagagccacacttggcccaggtagttgcagaagaccttctttcccccagtgtgatggatgtgggtgacttcaagatatcaatcaacgagggtcttccctctggggtgccctgcacctcgcaatggaactccatcacccactggctcctcactctttgtgcactctctgaagtcacggacctgtcccctgacatcattcaagccaattccttattctctttctatgg

SEQ ID NO: 104 - DNA, 382 bp, Bristol Virus
tcagagttggcatgaatatgaatgaggatggccccatcatcttcgagagacactccagatacaagtatcactatgatgctgactactctcggtgggattcaacacaacaaagggccgtgttagcagcagccctagaaatcatggttaaattctccccagaaccgcatttggcccagatagttgcagaagaccttctatctcctagtgtgatggatgtgggtgacttcaaaatatcaatcaatgagggccttccctctggtgtgccctgcacctctcaatggaattccatcgcccactggctcctcactctctgtgcactctctgaagttacaaacctgtcccctgacatcatacaggctaattccctcttttccttctatgg

SEQ ID NO: 105 - DNA, 18 bp, Artificial Sequence
  eubacterial primer A1.1
gaggcagcagtggggaat

SEQ ID NO: 106 - DNA, 47 bp, Artificial Sequence
  eubacterial primer S9T7
taatacgactcactatagggaggaggattactaccagggtatctaat

SEQ ID NO: 107 - DNA, 25 bp, Artificial Sequence
  primer XIA2F
ggaagggttgtattattagataaag

SEQ ID NO: 108 - DNA, 52 bp, Artificial Sequence
  primer XIA2R-T7
taatacgactcactatagggaggaggattaaaggagtaaggaacaacctcca

(19) United States
(12) Patent Application Publication
(10) Pub. No.: US 2008/0171314 A1
(43) Pub. Date: Jul. 17, 2008

(54) Method for controlling the microbiological quality of an aqueous medium and kit therefor

(75) Inventors: Patricia Renaud, Le Pecq (FR); Emmanuelle Guillot, Saint Germain En Laye (FR); Claude Mabilat, Saint Germain Au Mont D'or (FR); Carole Vachon, Villeurbanne (FR); Bruno Lacroix, Saint Genis Laval (FR); Guy Vernet, Albigny Sur Saone (FR); Marie-Astrid Armand, Charvieu Chavagneux (FR); Philippe Laffaire, Tignieu Jameyzieu (FR)

Correspondence Address: OLIFF & BERRIDGE, PLC, P.O. BOX 320850, ALEXANDRIA, VA, 22320-4850, US

(73) Assignee: BIOMERIEUX, Marcy L'Etoile (FR)

(21) Appl. No.: 12/010,716

(22) Filed: Jan. 29, 2008

Related U.S. Application Data
(62) Division of application No. 10/332,123, filed on Sep. 24, 2003, now abandoned.

(30) Foreign Application Priority Data
Jul. 6, 2000 (FR) 00.08839

(51) Int. Cl. C12Q 1/68; C07H 21/04; C12Q 1/70

(52) U.S. Cl. 435/5; 536/24.32; 435/6

(57) ABSTRACT

Disclosed is a method for controlling the microbiological quality of an environmental aqueous medium, suspected of containing various micro-organisms, having the following steps: selecting a reference set, having at least three micro-organisms, representing jointly or separately a microbiological quality level; providing a microbiological detection kit, having at least three probes specifically and respectively identifying said three micro-organisms; after treating the medium to be analyzed, contacting said micro-organisms, or any fraction thereof derived from the medium to be analyzed therefrom, with said detection kit, whereby a multiple determination of the micro-organisms is carried out, the determination representing the microbiological quality level of the medium.

This is a Division of application Ser. No. 10/332,123 filed Jan. 29, 2008, which in turn is a National Phase of Application No. PCT/FR01/02191 filed Jul. 6, 2001. The disclosures of the prior applications are hereby incorporated by reference herein in their entireties.

### BACKGROUND

The present invention falls within the the field of microbiological diagnosis, of detection techniques for identifying and quantifying microorganisms present in fluids and products, such as, for example, water.

It also relates to assaying kits and methods which make it possible to carry out these identifications and quantifications of microorganisms on samples with large volumes, in a time of less than a day, and which optionally allow production-monitoring control, or even a servo-control of the purification and production techniques by the results of these assays.

Conventional methods of microbiological identification require a step of culturing on selected media, in general followed by identification according to morphological, biochemical and/or immunological characteristics.

These methods are long, one day to several weeks for slow-growing bacteria, for example 10 to 12 days for Legionella, up to one month for mycobacteria, are relatively nonspecific, and are relatively insensitive when they are applied to a complex polymicrobial sample (water, environment, foods). In addition, they do not make it possible to detect viable but nonculturable (VBNC) bacteria stressed by environmental factors or disinfection treatments, and are not suitable for automation.

For more than ten years, molecular biology methods, in particular those based on enzymatic amplification in vitro (PCR) and the use of oligonucleotide probes, have revolutionized microbiological diagnosis.

Due to their rapidity, sensibility and specificity, they constitute an alternative to the conventional methods for detecting particular indicator or pathogenic microorganisms in samples of water or any sample, making it possible to detect the presence of such microorganisms in the environment.

Among the molecular biology methods used to detect in particular indicator or pathogenic microorganisms in samples of water or any sample, making it possible to detect the presence of such microorganisms in the environment, mention may particularly be made of the following.

To detect indicators of fecal contamination (total, thermotolerant coliforms, E coli) usually sought in the sanitary control of water, rapid assays based on a PCR-hybridization with a probe have been developed for drinking-water samples, in particular [A. K. Bej et al. Appl. Environ. Microbiol, 1990, No. 56, p. 307-314] [E. J. Fricker et al., Letters in Applied Microbiology, 1994, No. 19, p. 44-46].

These indicators of fecal contamination do not, however, make it possible to predict the presence of bacterial contamination of nonfecal origin (Pseudomonas aeruginosa, Legionella, etc.) or nonbacterial contaminations (viruses and parasites).

PCR-based molecular detection assays for specifically searching for pathogenic microorganisms (bacteria, viruses, parasites) have therefore been developed.

In the field of bacterial detection, European patent EP-A-0 438 115 will in particular be noted, which describes a method for detecting Legionella pathogenic microorganisms and fecal contamination indicators, via a step of in vitro enzymatic amplification in aquatic environmental samples.

Several publications also refer to PCR assays for detecting salmonella in water and the environment [J. S. Way et al., Appl. Environm. Microbiol., 1993, No. 59, p. 1473-1479] [A. S. Waage, et al., Appl. Microbiol., 1999, No. 87, p. 418-428], and also Legionella [A. K. Bej, Appl. Environ. Microbiol., 1991, No. 57, p. 2429-2432], and U.S. Pat. No. 5,298,392 describes the detection of fecal contamination indicators and pathogens.

In the field of viral detection, since the presence of viruses does not correlate with that of the fecal contamination indicators conventionally sought in the sanitary control of water, rapid and effective analytical methods are necessary, in particular for controlling viral contaminations of water.

The conventional methods for detecting viruses in water and the environment require a step of animal cell culture, a method which is long, cumbersome and restrictive, limited to a few viral families.

Many methods based on a step of enzymatic amplification have been described in order to search for pathogenic viruses in water and the environment. By way of examples, mention may be made, for detection by RT-PCR, of enteroviruses, hepatitis A and rotaviruses, in water samples [M. Abbaszadegan et al., Appl. Environ. Microbiol., 1997, No. 63(1), p. 324-328] and [M. Gilgen et al., International Journal of food Microbiology, 1997, No. 37, p. 189-199].

In the field of parasite detection, in particular for detecting Giardia and Cryptosporidium, which are two parasites whose transmission in water and the environment in an encysted form (oocyst and cyst) makes them particularly resistant to conventional treatments of disinfection such as chloration, conventional standardized methods (EPA 1622-1623 and DWI) have been developed. They comprise a filtration step followed by immunomagnetic capture (IMS) of oocysts and detection by immunofluorescence (IFA). These methods are long and fastidious, are not specific for species which are pathogenic for humans (Giardia lamblia and Cryptosporidium parvum) and do not make it possible to determine the viability of the parasites detected.

Molecular methods which are more rapid, sensitive and specific, based on an enzymatic amplification step (PCR), have been described.

WO-A-94/02635, WO-A-97/02281 and U.S. Pat. No. 5,693,472 describe primers and probes for detecting the species C. parvum in aquatic and/or biological samples.

EP-A-0 453 290 and U.S. Pat. No. 5,558,989 describe a method for detecting the species Giardia lamblia, which is pathogenic in humans, based on the use of nucleic acid (DNA and/or RNA) probes corresponding to the sequence of 18S rRNA. EP-A-0 550 883 describes a PCR assay with reagents for searching for G. lamblia, the sensitivity of which is 1-5 oocysts/ml of water concentrate.

Molecular methods which distinguish between dead parasites and viable and/or infectious parasites, thus making it possible to obtain a better assessment of the real sanitary risk posed by the presence of these parasites in water, have been described.

Mention will in particular be made of WO-A-97/42349, which relates to the detection of viable (by detecting hsp 70 heat shock protein mRNAs) and/or infectious (cell culture and enzymatic amplification) Cryptosporidium and Giardia, and U.S. Pat. No. 5,556,774 which relates to a method for detecting viable Cryptosporidium by combination of a PCR step and an in vitro excystation step.

While the main molecular methods cited above for searching for contamination indicators and pathogenic microorganisms including bacteria, parasites and viruses are much more effective than the conventional methods in terms of rapidity, sensitivity and specificity, they only target one type of microorganism per assay.

Thus, in order to measure or detect several parameters, it would be necessary to carry out as many specific assays as there are parameters to be measured or detected, which makes a complete microbiological analysis extremely laborious.

Some multidetection approaches have been described, but their capacity for multidetection is low since they detect only a maximum of 3 parameters. Mention will in particular be made of the multiplex PCR technique, which consists in carrying out several PCR reactions in the same tube.

By way of example, in [A. K. Bej et al., Appl. Environ. Microbiol., 1991, No. 57, p. 597-700], the simultaneous detection of Legionella and L. pneumophila and simultaneous detection on E. coli, Salmonella and Shigella are described, in [A. K. Bej et al., Appl. Environ. Microbiol., 1991, No. 57, p. 2429-2432] the simultaneous detection of total coliforms, E coli and Shigella is described, and in EP-A-0 438 115, the detection of Legionella and fecal contamination indicators is described.

The in situ hybridization (FISH) technique carried out with two or a maximum of three fluorescent probes can make it possible to detect several parameters simultaneously, but with a lower sensitivity than the enzymatic amplification methods methods mentioned above.

In the publication [M. Eggers et al., Presented at the 27^{th} International Conference on Environmental Systems, 1997], an approach is described for simultaneously detecting microorganisms in water and air, in space. This approach targets only bacteria, for example E. coli and Vibrio proteolyticus, by direct hybridization of 16S rRNA on a solid support (96-well microplate). There is no enzymatic amplification step and so the sensitivity is not very high, and the capacity for multidetection is restricted to a few microorganisms; however, a method of multidetection in water and air using a technique related to biochips is described.

### SUMMARY

Before continuing, and in the interests of clarity and clear understanding, various terms used in the description and claims need to be defined.

A “nucleotide fragment”, or an “oligonucleotide”, or a “polynucleotide”, is a chain of nucleotide motifs assembled together via phosphoric ester bonds, characterized by the informational sequence of natural nucleic acids capable of hybridizing to a nucleotide fragment under predetermined conditions, it being possible for the chain to contain monomers with different structures, and to be obtained from a natural nucleic acid molecule and/or by genetic recombination and/or by chemical synthesis.

A “nucleotide motif” is a derivative of a monomer, which maybe a natural nucleotide of nucleic acid, the constitutive elements of which are a sugar, a phosphate group and a nitrogenous base; in DNA, the sugar is 2-deoxyribose, in RNA, the sugar is ribose; depending on whether it is a question of DNA or RNA, the nitrogenous base is chosen from adenine, guanine, uracil, cytosine and thymine; or else the monomer is a nucleotide modified in at least one of the three constitutive elements mentioned above; by way of example, the modification may occur either at the level of the bases, with modified bases such as inosine, 5-methyldeoxycytidine, deoxyuridine, 5-dimethylaminodeoxyuridine, 2,6-diaminopurine, 5-bromodeoxyuridine or any other modified base capable of hybridization, or at the level of the sugar, for example replacement of at least one deoxyribose with a polyamide [P. E. Nielsen et al, Science, 1991, No. 254, p. 1497-1500], or else at the level of the phosphate group, for example replacement thereof with esters in particular chosen from diphosphates, alkyl- and arylphosphonates and phosphorothioates.

The term “informational sequence” is intended to mean any ordered series of motifs of the nucleotide type, the chemical nature of which and the order of which in a reference direction constitute information of the same quality as that of the natural nucleic acids.

The term “hybridization” is intended to mean the process during which, under suitable conditions, two nucleotide fragments having sufficiently complementary sequences are capable of forming a double strand with stable, specific hydrogen bonds. A nucleotide fragment “capable of hybridizing” with a polynucleotide is a fragment which can hybridize with said polynucleotide under hybridization conditions which can be determined, in each case, in a known manner. The hybridization conditions are determined by stringency, i.e. the severity of the operaton conditions. The higher the stringency at which the hybridization is carried out, the more specific it is. The stringency is defined in particular as a function of the composition of bases of a probe/target duplex, and also by the degree of mismatching between two nucleic acids.

The stringency can also depend on the parameters of the reaction, such as the concentration and the type of ionic species present in the hybridization solution, the nature and the concentration of denaturing agents and/or the hybridization temperature. The stringency of the conditions under which a hybridization reaction must be carried out will depend mainly on the probes used. All these data are well known and the suitable conditions can be determined by those skilled in the art.

In general, depending on the length of the probes used, the temperature for the hybridization reaction is between approximately 20 and 65° C., in particular between 35 and 65° C., in a saline solution at a concentration of approximately 0.8 to 1 molar.

A “probe” is a nucleotide fragment comprising from 5 to 100 monomers, in particular from 6 to 35 monomers, having a specificity of hybridization under given conditions so as to form a hybridization complex with a nucleotide fragment having, for example, a nucleotide sequence included in a ribosomal RNA, the DNA obtained by reverse transcription of said ribosomal RNA and the DNA (referred to herein as ribosomal DNA or rDNA) from which said ribosomal RNA is produced by transcription; a probe can be used for diagnostic purposes (in particular capture probes or detection probes).

A capture probe is immobilized, or can be immobilized, on a solid support by any suitable means, i.e. directly or indirectly, for example by covalence or adsorption.

A detection probe can be labeled using a label chosen from radioactive isotopes, enzymes (in particular a peroxydase, an alkaline phosphatase or an enzyme capable of hydrolyzing a chromogenic, fluorigenic or luminescent substrate), chromophoric chemical compounds, chromogenic, fluorogenic or luminescent compounds, nucleotide base analogs, and ligands such as biotin.

A “primer” is a probe comprising from 5 to 100, preferentially from 10 to 40, nucleotide motifs and having a specificity of hybridization under given conditions for the initiation of an enzymatic polymerization, for example, in an amplification technique such as PCR (Polymerase Chain Reaction), in a sequencing method, in a reverse transcription method, etc.

The identity between a fragment and a reference sequence, which characterizes the degree of identity between said fragment and said sequence, is measured by aligning said fragment on said sequence, and then determining the number of monomers which are identical between the two.

The probes and primers according to the invention are chosen from:

(a) the sequences identified in the sequence listing attached to the description, (b) any fragment of the sequences (a), both comprising at least 5 contiguous monomers included in any one of the sequences (a), and having a sequence exhibiting at least 70% identity with said sequence (a) ; by way of example, a fragment (b) comprises 10 nucleotides, among which 5 contiguous nucleotides belong to a sequence (a) and at least 2 nucleotides of the 5 remaining nucleotides are identical respectively to the two corresponding nucleotides in the reference sequence, after alignment.

The term “identifying sequence” denotes any sequence or any fragment as defined above which may be used as a detection and/or capture probe.

The expression “treatment of the aqueous medium” is intended to mean any filtration and/or lysis and/or purification step.

The term “lysis step” is intended to mean a step capable of releasing the nucleic acids contained in the protein and/or lipid envelopes of microorganisms (such as cell debris which interfere with subsequent reactions). By way of example, use may be made of the lysis methods as described in the applicant's patent applications:
WO-A-00/05338 on mixed magnetic and mechanical lysis,
WO-A-99/53304 on electrical lysis, and
   WO-A-99/15321 on mechanical lysis.
      Those skilled in the art may use other well-known methods of lysis, such as thermal or osmotic shocks or chemical lyses with chaotropic agents such as guanidium salts (U.S. Pat. No. 5,234,809).

The term “purification step” is intended to mean separation between the nucleic acids of the microorganisms and the cellular constituents released in the lysis step. This step generally makes it possible to concentrate the nucleic acids. By way of example, it is possible to use magnetic particles optionally coated with oligonucleotides, by adsorption or covalence (in this regard, see patents U.S. Pat. No. 4,672,040 and U.S. Pat. No. 5,750,338), and thus to purify the nucleic acids which have attached to these magnetic particles, via a washing step. This step of purification of the nucleic acids is particularly advantageous if subsequent amplification of said nucleic acids is desired. A particularly advantageous embodiment of these magnetic particles is described in the patent applications filed by the applicant under the following references: WO-A-97/45202 and WO-A-99/35500.

In the latter of these patent applications, it involves thermosensitive magnetic particles each having a magnetic core covered with an intermediate layer. The intermediate layer is itself covered with an outer layer based on a polymer capable of interacting with at least one biological molecule; the outer polymer is thermosensitive and has a predetermined lower critical solution temperature (LCST) of between 10 and 100° C., and preferably between 20 and 60° C. This outer layer is synthesized from cationic monomers which generate a polymer having the ability to bind nucleic acids. This intermediate layer isolates the magnetic charges of the core, in order to avoid problems of inhibition of techniques for amplifying these nucleic acids.

Another advantageous example of a method for purifying nucleic acids is the use of silica, either in the form of a column (Qiagen kits for example), or in the form of inert particles [R. Boom et al., J. Clin. Microbiol., 1990, No. 28(3), p. 495-503] or magnetic particles (Merck: MagPrep® Silica, Promega: MagneSil™ Paramagnetic particles). Other, very widely used methods are based on ion exchange resins in a column (Qiagen kits for example) or in a paramagnetic particulate format (Whatman: DEAE-Magarose) [PR Levison et al., J. Chromatography, 1998, p. 337-344]. Another method very relevant to the invention is that of adsorption onto a metal oxide support (company Xtrana: Xtra-Bind™ matrix).

The term “detection step” is intended to mean either direct detection by a physical method, or a method of detection using a label.

Many detection methods exist for detecting nucleic acids [see, for example, Kricka et al., Clinical Chemistry, 1999, No. 45(4), p. 453-458 or G. H. Keller et al., DNA Probes, 2nd Ed., Stockton Press, 1993, sections 5 and 6, p. 173-249].

In a first embodiment of the invention, a method of hybridization with specific probes is used for the detection step. This particular embodiment consists in bringing the nucleic acids, which may or may not be amplified, of the microorganisms to be detected in contact with a capture probe attached to a solid support and capable of hybridizing specifically with said nucleic acids; and then in revealing, according to known methods, the possible presence of the nucleic acids attached to the solid support in particular via at least one capture probe.

The term “label” is intended to mean a tracer capable of engendering a signal. A nonlimiting list of these tracers comprises enzymes which produce a signal which is detectable, for example by colorimetry, fluorescence or luminescence, such as horseradish peroxidase, alkaline phosphatase, beta-galactosidase or glucose-6-phosphate dehydrogenase; chromophores, such as fluorescent, luminescent or dye compounds; electron-dense groups detectable by electron microscopy or via their electrical properties such as conductivity, by amperometric or voltammetric methods, or by impedence measurements; groups detectable by optical methods such as diffraction, surface plasmon resonance or contact angle variation, or by physical methods such as atomic force spectroscopy, tunnel effect, etc.; radioactive molecules such as ³²p, ³⁵S or ¹²⁵I.

First, the polynucleotide may be labeled during the enzymatic amplification step, for example by using a labeled nucleotide triphosphate for the amplification reaction. The labeled nucleotide will be a deoxyribonucleotide in amplification systems generating a DNA, such as PCR, or a ribonucleotide in amplification techniques generating an RNA, such as the TMA or NASBA techniques.

The polynucleotide may also be labeled after the amplification step, for example by hybridizing a labeled probe according to the sandwich hybridization technique described in document WO-A-91/19812.

Another particular preferential method of labeling nucleic acids is described in the applicant's application FR-A-2 780 059. Another preferential detection method uses the 5′-3′ exonuclease activity of a polymerase as described by P. M. Holland, PNAS (1991) p 7276-7280.

Signal amplification systems can be used, as described in document WO-A-95/08000 and, in this case, the preliminary enzymatic amplification reaction may not be necessary.

The term “enzymatic amplification” is intended to mean a process generating multiple copies of a particular nucleotide fragment using specific primers, by the action of at least one enzyme. Thus, for amplifying nucleic acids, there exist, inter alia, the following techniques:

PCR (Polymerase Chain Reaction) as described in patents U.S. Pat. No. 4,683,195, U.S. Pat. No. 4,683,202 and U.S. Pat. No. 4,800,159,

LCR (Ligase Chain Reaction), reported, for example, in patent application EP-A-0201 184,

RCR (Repair Chain Reaction), described in patent application WO-A-90/01069,

3SR (Self Sustained Sequence Replication) with patent application WO-A-90/06995,

NASBA (Nucleic Acid Sequence-Based Amplification) with patent application WO-A-91/02818, and

TMA (Transcription Mediated Amplification) with patent U.S. Pat. No. 5,399,491.

The term “amplicons” is then used to denote the polynucleotides generated by an enzymatic amplification technique.

The term “solid support” as used herein includes all the materials on which a nucleic acid may be immobilized. Synthetic materials or natural materials, optionally chemically modified, may be used as a solid support, in particular polysaccharides, such as materials based on cellulose, for example paper, cellulose derivatives, such as cellulose acetate and nitrocellulose, or dextran; polymers, copolymers, in particular based on monomers of the styrene type, natural fibers such as cotton, and synthetic fibers such as nylon; inorganic materials such as silica, quartz, glasses, ceramics; latexes; magnetic particles; metal derivatives, gels, etc. The solid support may be in the form of a microtitration plate, of a membrane as described in application WO-A-94/12670, of a particle or of a biochip.

The term “biochip” is intended to mean a solid support small in size, on which are attached multiple capture probes at predetermined positions.

By way of illustration, examples of these biochips are given in the publicatons by [G. Ramsay, Nature Biotechnology, 1998, No. 16, p. 40-44; F. Ginot, Human Mutation, 1997, No. 10, p. 1-10; J. Cheng et al, Molecular diagnosis, 1996, No. 1(3), p. 183-200; T. Livache et al, Nucleic Acids Research, 1994, No. 22(15), p. 2915-2921; J. Cheng et al, Nature Biotechnology, 1998, No. 16, p. 541-546] or in patents U.S. Pat. No. 4,981,783, U.S. Pat. No. 5,700,637, U.S. Pat. No. 5,445,934, U.S. Pat. No. 5,744,305 and U.S. Pat. No. 5,807,522.

The main characteristic of the solid support should be that it conserves the characteristics of hybridization of the capture probes to the nucleic acids, while at the same time generating a minimum background noise for the detection method. An advantage of biochips is that they simplify the use of numerous capture probes, thus allowing multiple detection of microorganisms to be detected, while at the same time taking into account the polymorphism of said microorganisms to be detected.

The invention described hereinafter makes it possible to solve the problems posed by the methods previously described, equally in terms of sensitivity, specificity and capacity for multidetection, while at the same time being rapid and easy to implement.

A first subject of the invention is a method for controlling the microbiological quality of an aqueous environmental medium, liable to comprise various microorganisms, comprising the following steps:

a reference set, consisting of at least three microorganisms, representative, together or separately, of a level of microbiological quality, is chosen, a kit for microbiological determination is available, consisting of at least three identifying probes specifically and respectively for said three microorganisms, after treatment of the medium to be analyzed, said microorganisms, or any fraction obtained from the latter, are brought into contact with said determination kit as a result of which, said microorganisms are multidetermined, this determination being representative of the level of microbiological quality of the medium.

The invention also relates to a kit for microbiological determination, comprising a mixture of identifying probes for bacteria and/or for viruses and/or for parasites, said identifying probes each being specific for a bacterial, viral or parasite species or at least genus liable to be present in a sample of liquid to be assayed.

According to the invention, a kit denotes any manual, semi-automatic or automatic method for implementing an assaying means, the term “assaying” meaning identification and/or determination of viability and/or quantification, each of these three parameters being determined in sequence or according to the combinations: identification alone; identification and quantification; identification and viability; identification, quantification and viability.

This invention also relates to a method of multidetection using in particular biochip technology to search for a large number of microbiological parameters, including contamination indicators required in various legislations (USA, France, Europe) and pathogenic microorganisms, including bacteria, viruses and parasites.

In a single implementation, a complete microbiological analysis of a sample can be carried out with rapidity in, for example, approximately 4 hours, and with great sensitivity, for example of the order of 1 micro-target/101-1001 by virtue of the enzymatic amplification step.

This method of multidetection is specific for the species being sought by virtue of the use of sequences, termed identifying for sequences each species, as a probe, and can make it possible to determine the viability of the microorganisms by detecting viability markers such as, for example, rRNA and/or mRNA.

The rapidity, sensitivity and specificity of this method of multidetection make it possible to apply it equally to any aqueous environmental medium, i.e. any aqueous medium with the exclusion of any body fluid. In particular, this method applies to any water intended for human consumption, industrial clean water, urban and industrial residuary water, water from the agrofoods industry and water from processing, and to any fluid or product.

This simultaneous detection, in a single step, of multiple specific amplification products is possible through the use of a solid support in particular in the form of a solid support small in size to which are attached multiple capture probes at predetermined positions, or a “biochip”, these capture probes consisting of a set of fragments of or of entire specific nucleotide sequences, termed identifying sequences, for the microorganisms being sought.

These identifying sequences or these fragments can also be used in any known hybridization techniques, such as DOT-BLOT techniques [Maniatis et al, Molecular Cloning, Cold Spring Harbor, 1982], SOUTHERN BLOT techniques [E. M. Southern, J. Mol. Biol., 1975, 98, 503], NORTHERN BLOT techniques, or SANDWICH techniques [A. R. Dunn et al., Cell, 1977, 12,23].

Among the microorganisms sought, mention will be made, by way of example, to the following microorganisms:

Among Bacteria:

Escherichia coli, Escherichia coli SEROTYPE 0157:H7, Helicobacter pylori, Enterococcus faecalis, Enterococcus faecium, Enterococcus durans, Enterococcus hirae, Streptococcus bovis, Streptococcus equinus, Clostridium perfringens, Staphylococcus epidermatitis, Staphylococcus aureus, Campylobacter coli, Campylobacter jejuni, Aeromonas hydrophila, Aeromonas caviae, Aeromonas sobria, Pseudomonas aeruginosa, Vibrio cholerae, Acinetobacter baumanii, Burkholderia gladioli, Burkholderia cepacia, Stenotrophomonas maltophilia, the Mycobacterium genus, Mycobacterium avium, Mycobacterium intracellulare, Mycobacterium simiae, Mycobacterium kansasii, Mycobacterium xenopi, Mycobacterium marinum, Mycobacterium gordonae, the Legionella genus, Legionella pneumophila, the Salmonella genus.

Among viruses, and more particularly among Adenoviruses, Adenovirus 40, and Adenovirus 41a; Astroviruses, HastV-1-2; Enteroviruses, such as Poliovirus, Coxsackievirus, or Echovirus; Rotaviruses; Caliciviruses, such as Norwalk virus; Sapporo virus; and Hepatitis viruses such as Hepatitis A virus,

Among Parasites:

The Cryptosporidium genus, such as Cryptosporidium parvum, the Giardia genus, such as Giardia lamblia and Microsporidia.

The microorganisms can be sought at the level of the genus to which they belong, either at the lower taxonomic level, i.e. at the species level, or at the serotype and subtype level, and by epidemiology: for example for Legionella, the determination may be made with the identifying sequence SEQ ID NO:9 for the search at the genus level, and with SEQ ID NO: 10 or 11 for a determination with an identifying sequence specific for the bacterium Legionella pneumophila.

The sequences produced on the biochip, termed identifying sequences corresponding to the species being sought, will be chosen from the sequences, the list of which is attached in the annex, of SEQ ID NO:1 to SEQ ID NO:104.

Variants of implementation of the method according to the invention are set out hereinafter.

The kit for microbiological determination exposed to the microorganisms of the aqueous medium advantageously corresponds to any one of the following presentations:

The three identifying probes which it comprises have at least one sequence chosen from any one of the sequences SEQ ID Nos:1-104, and any fragments thereof comprising at least 5 contiguous monomers included in any one of said sequences, and having a sequence exhibiting at least 70% identity with said any sequence.

It comprises at least one identifying probe specific for a bacterium, at least one identifying probe specific for a parasite and at least one identifying probe specific for a virus; preferably, it comprises at least one identifying probe chosen from SEQ ID NO: 1 to SEQ ID NO:39, SEQ ID NO:62, SEQ ID NO:61, SEQ ID NO:66 to SEQ ID NO:69 and any fragments thereof comprising at least 5 contiguous monomers included in any one of said sequences, and having a sequence exhibiting at least 70% identity with said any sequence; at least one identifying probe chosen from SEQ ID NO:40 to SEQ ID NO:49, SEQ ID NO:63 to SEQ ID NO:65, and any fragments thereof comprising at least 5 contiguous monomers included in any one of said sequences, and having a sequence exhibiting at least 70% identity with said any sequence; and at least one sequence chosen from SEQ ID NO:50 to SEQ ID NO:60, and SEQ ID NO:70 to SEQ ID NO:104 and any fragments thereof comprising at least 5 contiguous monomers included in any one of said sequences, and having a sequence exhibiting at least 70% identity with said any sequence.

It comprises at least four identifying probes specific for at least four different bacteria; preferably, they are chosen from SEQ ID NO: 1 to SEQ ID NO:39, SEQ ID NO:62, SEQ ID NO:61, SEQ ID NO:66 to SEQ ID NO:69 and any fragments thereof comprising at least 5 contiguous monomers included in any one of said sequences, and having a sequence exhibiting at least 70% identity with said any sequence.

It comprises at least five identifying probes specific for at least five different viruses; preferably, they are chosen from SEQ ID NO:50 to SEQ ID NO:60 and SEQ ID NO:70 to SEQ ID NO: 104, and any fragments thereof comprising at least 5 contiguous monomers included in any one of said sequences, and having a sequence exhibiting at least 70% identity with said any sequence.

The kit for microbiological determination comprises at least two identifying probes specific for at least two parasites; preferably, it is chosen from SEQ ID NO:40 to SEQ ID NO:49 and SEQ ID NO:63 to SEQ ID NO:65, and any fragments thereof comprising at least 5 contiguous monomers included in any one of said sequences, and having a sequence exhibiting at least 70% identity with said any sequence.

The kit for microbiological determination comprises at least one identifying probe specific for a bacterium and at least one identifying probe specific for at least one parasite. Preferably, it comprises at least one identifying probe chosen from SEQ ID NO:1 to SEQ ID NO:39, SEQ ID NO:61, SEQ ID NO:62, SEQ ID NO:67 to SEQ ID NO:69, and any fragments thereof comprising at least 5 contiguous monomers included in any one of said sequences, and having a sequence exhibiting at least 70% identity with said any sequence, and at least one identifying probe included among SEQ ID NO:40 to SEQ ID NO:49, SEQ ID NO:63 to SEQ ID NO:65, and any fragments thereof comprising at least 5 contiguous monomers included in any one of said sequences, and having a sequence exhibiting at least 70% identity with said any sequence.

Said microorganisms of the kit for microbiological determination are chosen from the following bacteria: Escherichia coli, the Salmonella genus, Staphylococcus aureus. Preferably, at least one identifying probe of the kit is chosen from SEQ ID NO: 14, SEQ ID NO:62, SEQ ID NO9:66 [sic], SEQ ID NO:68, SEQ ID NO:69, SEQ ID NO:15, SEQ ID NO:23 and any fragments thereof comprising at least 5 contiguous monomers included in any one of said sequences, and having a sequence exhibiting at least 70% identity with said any sequence.

Said microorganisms of the kit for microbiological determination are chosen from the following bacteria: Escherichia coli, the Salmonella genus, Staphylococcus aureus, Clostridium perfringens. Preferably, at least one identifying probe of said kit is chosen from SEQ ID NO:14, SEQ ID NO:62, SEQ ID NO:66, SEQ ID NO:68, SEQ ID NO:69, SEQ ID NO:15, SEQ ID NO:23, SEQ ID NO:28, SEQ ID NO:29 and any fragments thereof comprising at least 5 contiguous monomers included in any one of said sequences, and having a sequence exhibiting at least 70% identity with said any sequence.

Said microorganisms of the kit for microbiological determination are chosen from the following microorganisms: Escherichia coli, the Salmonella genus, Staphylococcus aureus, the Cryptosporidium genus. Preferably, at least one probe of said kit is chosen from SEQ ID NO:14, SEQ ID NO:62, SEQ ID NO:66, SEQ ID NO:68, SEQ ID NO:69, SEQ ID NO:15, SEQ ID NO:23, SEQ ID NO:40 to SEQ ID NO:44 and any fragments thereof comprising at least 5 contiguous monomers included in any one of said sequences, and having a sequence exhibiting at least 70% identity with said any sequence.

Said microorganisms of the kit for microbiological determination are chosen from the following microorganisms: the Salmonella genus, Staphylococcus aureus, Giardia lamblia, Cryptosporidium parvum. Preferably, at least one probe of said kit is chosen from SEQ ID NO:15, SEQ ID NO:23, SEQ ID NO:46 to SEQ ID NO: 49, SEQ ID NO:63, SEQ ID NO:64 and SEQ ID NO:65, and any fragments thereof comprising at least 5 contiguous monomers included in any one of said sequences, and having a sequence exhibiting at least 70% identity with said any sequence.

Said microorganisms of the kit for microbiological determination are chosen from the following microorganisms: Escherichia coli, Enteroviruses, the Cryptosporidium genus. Preferably, at least one identifying probe of said kit is chosen from SEQ ID NO:14, SEQ ID NO:62, SEQ ID NO:66, SEQ ID NO:68, SEQ ID NO:69, SEQ ID NO:53 to SEQ ID NO:55, SEQ ID NO:70 to SEQ ID NO:75, SEQ ID NO:40 to SEQ ID NO:44, and any fragments thereof comprising at least 5 contiguous monomers included in any one of said sequences, and having a sequence exhibiting at least 70% identity with said any sequence.

Said microorganisms of the kit for microbiological determination are chosen from the following microorganisms: Escherichia coli, Escherichia coli serotype 0157:H7, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsackievirus A and B, Echovirus, the Cryptosporidium genus, Cryptosporidium parvum, the Giardia genus, Giardia lamblia. Preferably, at least one identifying probe of said kit is chosen from SEQ ID NO:14, SEQ ID NO:15, SEQ ID NO:23, SEQ ID NO:25 to SEQ ID NO:29, SEQ ID NO:40 to SEQ ID NO:49, SEQ ID NO:53 to SEQ ID NO:55, SEQ ID NO:61 to SEQ ID NO:75, and any fragments thereof comprising at least 5 contiguous monomers included in any one of said sequences, and having a sequence exhibiting at least 70% identity with said any sequence.

Said microorganisms of the kit for microbiological determination are chosen from the following microorganisms: Escherichia coli, Escherichia coli serotype 0157:H7, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsackievirus A and B, Echoviruses, the Cryptosporidium genus, Cryptosporidium parvum, the Giardia genus, Giardia lamblia, the Legionella genus, Legionella pneumophila, Aeromonas hydrophila, Aeromonas caviae, Aeromonas sobria, Campylobacter coli, Campylobacter jejuni, Hepatitis A virus, Caliciviruses: Norwalk and Sapporo Virus, Adenoviruses, Rotaviruses. Preferably, at least one identifying probe of the kit is chosen from SEQ ID NO:1 to SEQ ID NO:4, SEQ ID NO:9 to SEQ ID NO:11, SEQ ID NO:14 to SEQ ID NO:20, SEQ ID NO:23, SEQ ID NO:25 to SEQ ID NO:29, SEQ ID NO:40 to SEQ ID NO:51, SEQ ID NO:53 to SEQ ID NO:55, SEQ ID NO:56 to SEQ ID NO:104, and any fragments thereof comprising at least 5 contiguous monomers included in any one of said sequences, and having a sequence exhibiting at least 70% identity with said any sequence.

Said microorganisms of the kit for microbiological determination are chosen from the following microorganisms: Escherichia coli, Escherichia coli serotype 0157:H7, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsackievirus A and B, Echoviruses, the Cryptosporidium genus, Cryptosporidium parvum, the Giardia genus, Giardia lamblia, the Legionella genus, Legionella pneumophila, Aeromonas hydrophila, Aeromonas caviae, Aeromonas sobria, Campylobacter coli, Campylobacter jejuni, hepatitis A virus, Caliciviruses: Norwalk and Sapporo virus, Adenoviruses, Rotaviruses, Pseudomonas aeruginosa, Vibrio cholerae, the Mycobacterium genus, Mycobacterium avium, Mycobacterium intracellulare, Mycobacterium simiae, Mycobacterium kansasii, Mycobacterium xenopi, Mycobacterium marinum, Mycobacterium gordonae, Acinetobacter baumanii, Staphylococcus epidermidis, Burkholderia gladioli, Burkholderia cepacia, Stenotrophomonas maltophilia, Astroviruses. Preferably, at least one identifying probe of the determination kit is chosen from SEQ ID NO:1 to SEQ ID NO:6, SEQ ID NO:9 to SEQ ID NO:55, SEQ ID NO:56 to SEQ ID NO:104, and any fragments thereof comprising at least 5 contiguous monomers included in any one of said sequences, and having a sequence exhibiting at least 70% identity with said any sequence.

Said microorganisms of the kit for microbiological determination are chosen from the following bacteria: Escherichia coli, Escherichia coli SEROTYPE 0157:H7, Helicobacter pylori, Enterococcus faecalis, Enterococcus faecium, Enterococcus durans, Enterococcus hirae, Streptococcus bovis, Streptococcus equinus, Clostridium perfringens, Staphylococcus epidermitis, Staphylococcus aureus, Campylobacter coli, Campylobacter jejuni, Aeromonas hydrophila, Aeromonas caviae, Aeromonas sobria, Pseudomonas aeruginosa, Vibrio cholerae, Acinetobacter baumanii, Burkholderia gladioli, Burkholderia cepacia, Stenotrophomonas maltophilia, the Mycobacterium genus, Mycobacterium avium, Mycobacterium intracellulare, Mycobacterium simiae, Mycobacterium kansasii, Mycobacterium xenopi, Mycobacterium marinum, Mycobacterium gordonae, the Legionella genus, Legionella pneumophila, the Salmonella genus.

Said microorganisms of the kit for microbiological determination are chosen from the following viruses:
Adenoviruses, such as Adenovirus 40, Adenovirus 41a;
Astroviruses, HAstV-1-2;
Enteroviruses, such as Poliovirus, Coxsackievirus, Echovirus,
Rotaviruses,
Caliciviruses, such as Norwalk virus, Sapporo virus, and the hepatitis viruses such as the hepatitis A virus.

Said microorganisms of the kit for microbiological determination are chosen from the following parasites:

The Cryptosporidium genus, Cryptosporidium parvum, the Giardia genus, Giardia lamblia and Microsporidia.

Said microorganisms of the kit for microbiological determination are chosen from the following microorganisms: Escherichia coli, Escherichia coli serotype 0157:H7, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsackievirus A and B, Echoviruses, the Cryptosporidium genus, Cryptosporidium parvum, the Giardia genus, Giardia lamblia, the Legionella genus, Legionella pneumophila, Aeromonas hydrophila, Aeromonas caviae, Aeromonas sobria, Campylobacter coli, Campylobacter jejuni, hepatitis A virus. Preferably, at least one identifying probe of the kit is chosen from SEQ ID NO: 1 to SEQ ID NO:4, SEQ ID NO:9 to SEQ ID NO:11, SEQ ID NO:14 to SEQ ID NO:20, SEQ ID NO:23, SEQ ID NO:25 to SEQ ID NO:29, SEQ ID NO:40 to SEQ ID NO:51, SEQ ID NO:53 to SEQ ID NO:55, SEQ ID NO:56 to SEQ ID NO:75, SEQ ID NO:97, and any fragments thereof comprising at least 5 contiguous monomers included in any one of said sequences, and having a sequence exhibiting at least 70% identity with said any sequence.

Said microorganisms of the kit for microbiological determination are chosen from the following microorganisms: Escherichia coli, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsackievirus A and B, Echoviruses, the Cryptosporidium genus, Cryptosporidium parvum, the Giardia genus, Giardia lamblia, the Legionella genus, Legionella pneumophila, Aeromonas hydrophila, Aeromonas caviae, Aeromonas sobria, Campylobacter coli, Campylobacter jejuni, hepatitis A virus. Preferably, at least one identifying probe of the kit is chosen from SEQ ID NO: 1 to SEQ ID NO:4, SEQ ID NO:9 to SEQ ID NO:11, SEQ ID NO: 14 to SEQ ID NO:20, SEQ ID NO:23, SEQ ID NO:25 to SEQ ID NO:29, SEQ ID NO:40 to SEQ ID NO:51, SEQ ID NO:53 to SEQ ID NO:55, SEQ ID NO:56 to SEQ ID NO:68, SEQ ID NO:70 to SEQ ID NO:75, SEQ ID NO:97 and any fragments thereof comprising at least 5 contiguous monomers included in any one of said sequences, and having a sequence exhibiting at least 70% identity with said any sequence.

Said microorganisms of the kit for microbiological determination are chosen from the following bacteria:

Escherichia coli, Escherichia coli SEROTYPE 0157:H7, the Salmonella genus, Pseudomonas aeruginosa, the Mycobacterium genus, the Legionella genus, Legionella pneumophila, Staphylococcus aureus. Preferably, at least one identifying probe of the determination kit is chosen from SEQ ID NO: 14, SEQ ID NO 62, SEQ ID NO 66 to SEQ ID NO 69, SEQ ID NO 15, SEQ ID NO 5, SEQ ID NO 6, SEQ ID NO 30, SEQ ID NO 9 to SEQ ID NO 11, SEQ ID 23 and any fragments thereof comprising at least 5 contiguous monomers included in any one of said sequences, and having a sequence exhibiting at least 70% identity with said any sequence.

Said microorganisms of the kit for microbiological determination are chosen from the following viruses:

Hepatitis A virus, Enteroviruses and at least one virus chosen from Caliciviruses and Rotaviruses. Preferably, at least one identifying probe of the determination kit is chosen from SEQ ID NO 59, SEQ ID NO:60 SEQ ID NO 97, SEQ ID NO 70 to SEQ ID NO 96, SEQ ID NO:98 to SEQ ID NO: 104 and any fragments thereof comprising at least 5 contiguous monomers included in any one of said sequences, and having a sequence exhibiting at least 70% identity with said any sequence.

Said microorganisms of the kit for microbiological determination are chosen from the following viruses:

Hepatitis A virus, Enteroviruses, at least one virus chosen from the Norwalk virus and Rotaviruses. Preferably, at least one identifying probe of the determination kit is chosen from SEQ ID NO 98 to 104, SEQ ID NO 59, SEQ ID NO 56 to SEQ ID NO 58, SEQ ID NO:60, SEQ ID NO:97, SEQ ID NO:70 to SEQ ID NO:75, SEQ ID NO 76 to SEQ ID NO 96 and any fragments thereof comprising at least 5 contiguous monomers included in any one of said sequences, and having a sequence exhibiting at least 70% identity with said any sequence.

Said microorganisms of the kit for microbiological determination are chosen from the following parasites: the Cryptosporidium genus, Cryptosporidium parvum, the Giardia genus, Giardia Lamblia. Preferably, at least one identifying probe of the determination kit is chosen from SEQ ID NO:40 to SEQ ID NO 45, SEQ ID NO 65 and any fragments thereof comprising at least 5 contiguous monomers included in any one of said sequences, and having a sequence exhibiting at least 70% identity with said any sequence.

Said microorganisms of the kit for microbiological determination are chosen from:

Escherichia coli, Escherichia coli SEROTYPE 0157:H7, the Salmonella genus, Pseudomonas aeruginosa, the Mycobacterium genus, the Legionella genus, Legionella pneumophila, Staphylococcus aureus, hepatitis A virus, Enteroviruses, and at least one virus chosen from Caliciviruses and Rotaviruses, the Cryptosporidium genus, Cryptosporidium parvum, the Giardia genus, Giardia Lamblia; preferably, at least one identifying probe of the determination kit is chosen from SEQ ID NO:14, SEQ ID NO 62, SEQ ID NO 66 to SEQ ID NO 69, SEQ ID NO 15, SEQ ID NO 5, SEQ ID NO 6, SEQ ID NO 30, SEQ ID NO 9 to SEQ ID NO 11,SEQ ID 23,SEQ ID NO 59, SEQ ID NO:60 SEQ ID NO 97, SEQ ID NO 70 to SEQ ID NO 96, SEQ ID NO:98, SEQ ID NO:40 to SEQ ID NO 45, SEQ ID NO 65 and any fragments thereof comprising at least 5 contiguous monomers included in any one of said sequences, and having a sequence exhibiting at least 70% identity with said any sequence, or Escherichia coli, Escherichia coli SEROTYPE 0157:H7, the Salmonella genus, Pseudomonas aeruginosa, the Mycobacterium genus, the Legionella genus, Legionella pneumophila, Staphylococcus aureus, hepatitis A virus, Enteroviruses, and at least one virus chosen from the Norwalk virus and Rotaviruses, the Cryptosporidium genus, Cryptosporidium parvum, the Giardia genus, Giardia Lamblia; preferably, at least one identifying probe chosen from SEQ ID NO: 14, SEQ ID NO 62, SEQ ID NO 66 to SEQ ID NO 69, SEQ ID NO 15, SEQ ID NO 5, SEQ ID NO 6, SEQ ID NO 30, SEQ ID NO 9 to SEQ ID NO 11, SEQ ID 23, SEQ ID NO 98 to 104, SEQ ID NO 59, SEQ ID NO 56 to SEQ ID NO 58, SEQ ID NO:60, SEQ ID NO:97, SEQ ID NO:70 to SEQ ID NO:75, SEQ ID NO 76 to SEQ ID NO 96, SEQ ID NO:40 to SEQ ID NO 45, SEQ ID NO 65 and any fragments thereof comprising at least 5 contiguous monomers included in any one of said sequences, and having a sequence exhibiting at least 70% identity with said any sequence.

Said microorganisms of the kit for microbiological determination are chosen from Norwalk virus, hepatitis A virus, Enteroviruses. Preferably, at least one identifying probe chosen from SEQ ID NO 59, SEQ ID NO:60, SEQ ID NO 97, SEQ ID NO 70 to SEQ ID NO 75.

The capture probes advantageously comprise at least 10, preferably at least 13, or indeed at least 15, even at least 17, bases and/or at most 35, preferably at most 25, or indeed at most 20. For example, a capture probe comprises between 10 and 35 bases, advantageously between 17 and 20 bases, with at least one interrogation position located in the central region of the known sequence, at the 12th position relative to the 3′ end of the sequence. For the species E. coli and E. faecalis, there will preferably be capture probes of 17 bases, with 2 interrogation positions, one at the 10th position and one at the 8th position. These capture probes are between 10 and 25 nucleotides long, depending on the case. The interrogation positions then vary as a function of the length of the capture probe.

The specific sequences, termed identifying sequences, were selected by computer selection techniques and are each sufficiently specific for a species and/or for a member of a species to make it possible to distinguish taxonomically close genera and/or species of the same genus, and to avoid phenomena of cross hybridization.

In one embodiment of the invention, the kit for microbiological determination comprises a mixture of identifying probes for bacteria and/or for viruses and/or for parasites, comprising at least four identifying probes specific for at least four different bacteria.

In another embodiment of the invention, the kit for microbiological determination comprises a mixture of identifying probes for bacteria and/or for viruses and/or for parasites, comprising at least five identifying probes specific for at least five different viruses.

In another embodiment of the invention, the kit for microbiological determination comprises a mixture of identifying probes for bacteria and/or for viruses and/or for parasites, comprising at least two identifying probes specific for a parasite.

In another embodiment of the invention, the kit for microbiological determination comprises a mixture of identifying probes for bacteria and/or for viruses and/or for parasites, comprising at least one probe specific for a bacterium and at least one identifying probe specific for a parasite.

In another embodiment of the invention, the kit for microbiological determination comprises a mixture of identifying probes for bacteria and/or for viruses and/or for parasites, in which the probes specific for bacteria are chosen from the probes specific for the following bacteria:

Escherichia coli, the Salmonella genus, Staphylococcus aureus.

In another embodiment of the invention, the kit for microbiological determination comprises a mixture of identifying probes for bacteria and/or for viruses and/or for parasites, in which the probes specific for bacteria are chosen from the probes specific for the following bacteria:

Escherichia coli, the Salmonella genus, Staphylococcus aureus, Clostridium perfringens.

In another embodiment of the invention, the kit for microbiological determination comprises a mixture of identifying probes for bacteria and/or for viruses and/or for parasites, in which the probes specific for bacteria are chosen from the probes specific for the following bacteria:

Escherichia coli, Enterococcus faecalis, Enterococcus faecium, Enterococcus durans, Enterococcus hirae, Streptococcus bovis, Streptococcus equinus, Clostridium perfringens.

In another embodiment of the invention, the kit for microbiological determination comprises a mixture of identifying probes for bacteria and/or for viruses and/or for parasites, in which the probes specific for bacteria are chosen from the probes specific for the following bacteria:

Escherichia coli, Escherichia coli SEROTYPE 0157:H7, Helicobacter pylori, Enterococcus faecalis, Enterococcus faecium, Enterococcus durans, Enterococcus hirae, Streptococcus bovis, Streptococcus equinus, Clostridium perfringens, Staphylococcus epidermitis, Staphylococcus aureus, Campylobacter coli, Campylobacter jejuni, Aeromonas hydrophila, Aeromonas caviae, Aeromonas sobria, Pseudomonas aeruginosa, Vibrio cholerae, Acinetobacter baumanii, Burkholderia gladioli, Burkholderia cepacia, Stenotrophomonas maltophilia, the Mycobacterium genus, Mycobacterium avium, Mycobacterium intracellulare, Mycobacterium simiae, Mycobacterium kansasii, Mycobacterium xenopi, Mycobacterium marinum, Mycobacterium gordonae, the Legionella genus, Legionella pneumophila, the Salmonella genus.

In another embodiment of the invention, the kit for microbiological determination comprises a mixture of identifying probes for bacteria and/or for viruses and/or for parasites chosen from the following microorganisms:

Escherichia coli, the Salmonella genus, Staphylococcus aureus, the Cryptosporidium genus.

In another embodiment of the invention, the kit for microbiological determination comprises a mixture of identifying probes for bacteria and/or for viruses and/or for parasites chosen from the following microorganisms:

the Salmonella genus, Staphylococcus aureus, Giardia lamblia, Cryptosporidium parvum.

In another embodiment of the invention, the kit for microbiological determination comprises a mixture of identifying probes for bacteria and/or for viruses and/or for parasites chosen from the following microorganisms:

Escherichia coli, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsachievirus A and B, Echoviruses, the Cryptosporidium genus, Cryptosporidium parvum, Giardia lamblia,

In another embodiment of the invention, the kit for microbiological determination comprises a mixture of identifying probes for bacteria and/or for viruses and/or for parasites chosen from the following microorganisms:

Escherichia coli, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsackievirus A and B, Echoviruses, the Cryptosporidium genus, Cryptosporidium parvum, Giardia lamblia, the Legionella genus, Legionella pneumophila, Aeromonas hydrophila, Aeromonas caviae, Aeromonas sobria, Campylobacter coli, Campylobacter jejuni, hepatitis A virus, Caliciviruses: Norwalk and Sapporo virus, Adenoviruses, Rotaviruses.

In another embodiment of the invention, the kit for microbiological determination comprises a mixture of identifying probes for bacteria and/or for viruses and/or for parasites chosen from the following microorganisms:

Escherichia coli, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis, coxsackievirus A and B, Echoviruses, the Cryptosporidium genus, Cryptosporidium parvum, Giardia lamblia, the Legionella genus, Legionella pneumophila, Aeromonas hydrophila, Aeromonas caviae, Aeromonas sobria, Campylobacter coli, Campylobacter jejuni, hepatitis virus A, Caliciviruses: Norwalk and Sapporo virus, Adenoviruses, Rotaviruses, Pseudomonas aeruginosa, Vibrio cholerae, the Mycobacterium genus, Mycobacterium avium, Mycobacterium intracellulare, Mycobacterium simiae, Mycobacterium kansasii, Mycobacterium xenopi, Mycobacterium marinum, Mycobacterium gordonae, Acinetobacter baumanii, Staphylococcus epidermidis, Burkholderia gladioli, Burkholderia cepacia, Stenotrophomonas maltophilia, Astroviruses.

In another embodiment of the invention, the kit for microbiological determination comprises a mixture of identifying probes for bacteria and/or for viruses and/or for parasites chosen from the following microorganisms:

Escherichia coli, Enteroviruses, the Cryptosporidium genus.

In another embodiment of the invention, the kit for microbiological determination comprises a mixture of identifying probes for bacteria and/or for viruses and/or for parasites, in which the identifying probes specific for viruses are specific for the following viruses:

Adenoviruses, such as Adenovirus 40, Adenovirus 41a;

Astroviruses, HastV-1-2;

Enteroviruses, such as Poliovirus, Coxsackievirus, Echovirus,

Rota viruses,

Calicivirus, such as Norwalk virus, Sapporo virus and hepatitis viruses, such as hepatitis A virus.

In another embodiment of the invention, the kit for microbiological determination comprises a mixture of identifying probes for bacteria and/or for viruses and/or for parasites, in which the probes specific for parasites are chosen from the probes specific for the following parasites:

The Cryptosporidium genus, Cryptosporidium parvum, Giardia lamblia and Microsporidia.

According to the invention, in one embodiment, the kit for microbiological determination of a microorganism present in a sample comprises at least one identifying probe chosen from SEQ ID NO:1 to SEQ ID NO:39, SEQ ID NO:61, SEQ ID NO:62, SEQ ID NO:66 to SEQ ID NO:69 and any fragments thereof comprising at least 5 contiguous monomers included in any one of said sequences, and having a sequence exhibiting at least 70% identity with said any sequence; at least one identifying probe chosen from SEQ ID NO:40 to SEQ ID NO:49, SEQ ID NO: 63 to SEQ ID NO:65 and any fragments thereof comprising at least 5 contiguous monomers included in any one of said sequences, and having a sequence exhibiting at least 70% identity with said any sequence; and at least one sequence chosen from SEQ ID NO:50 to SEQ ID NO:60, SEQ ID NO:70 to SEQ ID NO:104 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least 4 identifying probes chosen from SEQ ID NO:1 to SEQ ID NO:39, SEQ ID NO:61, SEQ ID NO:62, SEQ ID NO:66 to SEQ ID NO:69 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least 5 identifying probes included among SEQ ID NO:50 to SEQ ID NO:60, SEQ ID NO:70 to SEQ ID NO: 104 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one identifying probe chosen from SEQ ID NO:40 to SEQ ID NO:49, SEQ ID NO: 63 to SEQ ID NO:65 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one identifying probe chosen from SEQ ID NO:1 to SEQ ID NO:39, SEQ ID NO:61, SEQ ID NO:62, SEQ ID NO:66 to SEQ ID NO:69 and any fragments thereof comprising at least 5 contiguous monomers included in any one of said sequences, and having a sequence exhibiting at least 70% identity with said any sequence, and at least one identifying probe chosen from SEQ ID NO:40 to SEQ ID NO:49, SEQ ID NO: 63 to SEQ ID NO:65 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one identifying probe chosen from SEQ ID NO:14, SEQ ID NO:62, SEQ ID NO:15, SEQ ID NO:23, SEQ ID NO:66, SEQ ID NO:68, SEQ ID NO:69 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one identifying probe chosen from SEQ ID NO:14, SEQ ID NO:62, SEQ ID NO:66, SEQ ID NO:68, SEQ ID NO:69, SEQ ID NO:15, SEQ ID NO:23, SEQ ID NO:28, SEQ ID NO:29 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one identifying probe chosen from SEQ ID NO:14, SEQ ID NO:62, SEQ ID NO:66, SEQ ID NO:68, SEQ ID NO:69, SEQ ID NO:15, SEQ ID NO:23, SEQ ID NO:40 to SEQ ID NO:44 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one identifying probe chosen from SEQ ID NO:14, SEQ ID NO:62, SEQ ID NO:66, SEQ ID NO:68, SEQ ID NO:69, SEQ ID NO:53 to SEQ ID NO:55, SEQ ID NO:70 to SEQ ID NO:75, SEQ ID NO:40 to SEQ ID NO:44 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one identifying probe chosen from SEQ ID NO:14, SEQ ID NO:15, SEQ ID NO:23, SEQ ID NO:25 to SEQ ID NO:29, SEQ ID NO:40 to SEQ ID NO:49, SEQ ID NO:53 to SEQ ID NO:55, SEQ ID NO:61 to SEQ ID NO:75 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one identifying probe chosen from SEQ ID NO: 1 to SEQ ID NO:4, SEQ ID NO:9, SEQ ID NO:10, SEQ ID NO:11, SEQ ID NO:14 to SEQ ID NO:20, SEQ ID NO:23, SEQ ID NO:25 to SEQ ID NO:29, SEQ ID NO:40 to SEQ ID NO:51, SEQ ID NO:53 to SEQ ID NO:55, SEQ ID NO:56 to SEQ ID NO:59, SEQ ID NO:60 to SEQ ID NO:65 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one sequence included among SEQ ID NO:1 to SEQ ID NO:6, SEQ ID NO:9 to SEQ ID NO:22, and SEQ ID NO:23 to SEQ ID NO:55, SEQ ID NO:56 to SEQ ID NO:104 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one sequence chosen from SEQ ID NO:14, SEQ ID NO 62, SEQ ID NO 66 to SEQ BD NO 69, SEQ ID NO 15, SEQ ID NO 5, SEQ ID NO6, SEQ ID NO30, SEQ ID NO9 to SEQ ID NO 11, SEQ ID NO 23 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one sequence chosen from SEQ ID NO 59, SEQ ID NO 97, SEQ ID NO 70 to SEQ ID NO 75 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one sequence chosen from SEQ ID NO 98 to 104, SEQ ID NO 59, SEQ ID NO 98, SEQ ID NO 56 to SEQ ID NO 58, SEQ ID NO 76 to SEQ ID NO 96 and any fragments thereof comprising at least 5 contiguous monomers included in any one of said sequences, and having a sequence exhibiting at least 70% identity with said any sequence.

According to the invention, in a different embodiment, the kit for microbiological determination of a microorganism present in a sample comprises at least one sequence chosen from SEQ ID NO:40 to SEQ ID NO 45, SEQ ID NO 65 and any fragments thereof comprising at least 5 contiguous monomers included in any one of said sequences, and having a sequence exhibiting at least 70% identity with said any sequence.

These nucleotide fragments, termed identifying sequences, according to the invention make it possible to selectively assay a microorganism in the presence of at least 2 other microorganisms chosen from the following microorganisms:

Escherichia coli, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsackievirus A and B, Echoviruses, the Cryptosporidium genus, Cryptosporidium parvum, Giardia lamblia, the Legionella genus, Legionella pneumophila, Aeromonas hydrophila, Aeromonas caviae, Aeromonas sobria, Campylobacter coli, Campylobacter jejuni, hepatitis A virus, Caliciviruses: Norwalk and Sapporo virus, Adenoviruses, Rotaviruses, Pseudomonas aeruginosa, Vibrio cholerae, the Mycobacterium genus, Mycobacterium avium, Mycobacterium intracellulare, Mycobacterium simiae, Mycobacterium kansasii, Mycobacterium xenopi, Mycobacterium marinum, Mycobacterium gordonae, Acinetobacter baumanii, Staphylococcus epidermidis, Burkholderia gladioli, Burkholderia cepacia, Stenotrophomonas maltophilia, Astroviruses;

Adenoviruses, such as Adenovirus 40, Adenovirus 41a;

Astroviruses, HastV-1-2;

Enteroviruses, such as Poliovirus, Coxsackievirus, Echovirus;

Rotaviruses;

Calicivirus, such as Norwalk virus, Sapporo virus and hepatitis viruses, such as hepatitis A virus; and

the Cryptosporidium genus, such as Cryptosporidium parvum, Giardia lamblia and Microsporidia.

In another embodiment, said identifying sequences according to the invention make it possible to selectively assay a microorganism in the presence of at least 2 other microorganisms chosen from the following microorganisms:

Escherichia coli, the Salmonella genus, Staphylococcus aureus.

In another embodiment, said identifying sequences according to the invention make it possible to selectively assay a microorganism in the presence of at least 2 other microorganisms chosen from the following microorganisms:

Escherichia coli, the Salmonella genus, Staphylococcus aureus, Clostridium perfringens.

In another embodiment, said identifying sequences according to the invention make it possible to selectively assay a microorganism in the presence of at least 2 other microorganisms chosen from the following microorganisms:

Escherichia coli, the Salmonella genus, Staphylococcus aureus, the Cryptosporidium genus.

In another embodiment, said identifying sequences according to the invention make it possible to selectively assay a microorganism in the presence of at least 2 other microorganisms chosen from the following microorganisms:

Escherichia coli, Enteroviruses and the Cryptosporidium genus.

In another embodiment, said identifying sequences according to the invention make it possible to selectively assay a microorganism in the presence of at least 2 other microorganisms chosen from the following microorganisms:

Escherichia coli, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, coxsackievirus A and B, Echoviruses, the Cryptosporidium genus, Cryptosporidium parvum, Giardia lamblia,

In another embodiment, the identifying sequences according to the invention make it possible to selectively assay a microorganism in the presence of at least 2 other microorganisms chosen from the following microorganisms:

Escherichia coli, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis virus, virus coxsackie A and B, Echoviruses, the Cryptosporidium genus, Cryptosporidium parvum, Giardia lamblia, the Legionella genus, Legionella pneumophila, Aeromonas hydrophila, Aeromonas caviae, Aeromonas sobria, Campylobacter coli, Campylobacter jejuni, hepatitis A virus, Calicivirus: Norwalk and Sapporo virus, Adenoviruses, Rota viruses.

In another embodiment, the identifying sequences according to the invention make it possible to selectively assay a microorganism in the presence of at least 2 other microorganisms chosen from the following microorganisms:

Escherichia coli, Enterococcus faecalis, Enterococcus faecium, Enterococcus durans, Enterococcus hirae, Streptococcus bovis, Streptococcus equinus, Clostridium perfringens, the Salmonella genus, Staphylococcus aureus, Enteroviruses: poliomyelitis, coxsackievirus A and B, Echoviruses, the Cryptosporidium genus, Cryptosporidium parvum, Giardia lamblia, the Legionella genus, Legionella pneumophila, Aeromonas hydrophila, Aeromonas caviae, Aeromonas sobria, Campylobacter coli, Campylobacter jejuni, hepatitis virus A, Caliciviruses: Norwalk and Sapporo virus, Adenoviruses, Rotaviruses, Pseudomonas aeruginosa, Vibrio cholerae, the Mycobacterium genus, Mycobacterium avium, Mycobacterium intracellulare, Mycobacterium simiae, Mycobacterium kansasii, Mycobacterium xenopi, Mycobacterium marinum, Mycobacterium gordonae, Acinetobacter baumanii, Staphylococcus epidermidis, Burkholderia gladioli, Burkholderia cepacia, Stenotrophomonas maltophilia, Astroviruses.

A kit for microbiological determination according to the invention, comprising identifier sequences for identifying microorganisms at the serotype and subtype level and by epidemiology, may also be designed and used.

The method for analyzing a sample liable to contain at least one bacterium, parasite and/or virus, according to the invention, uses a mixture of nucleotide sequences as identifying probes specific for a bacterial, viral and/or parasite serotype, subtype, species or at least one genus liable to be present in the sample.

This method for analyzing a sample according to the invention is characterized by a detection step comprising the use of a kit for microbiological determination as defined above.

In a preferential embodiment, prior to the detection step, at least one lysis step may be carried out.

In another embodiment, subsequent to this lysis step, an amplification step is carried out.

The invention also relates to a method for controlling a liquid sample, in which, prior to any detection step, a step of enriching said sample in microorganisms is carried out.

This enriching step may be carried out by filtration, in particular using a filtration means comprising hollow fibers and used in frontal mode, making it possible to obtain, in a limited period of time, from a starting liquid sample with a large and predetermined volume, a sample to be analyzed with a sufficiently small volume, while at the same time guaranteeing the viability of the microorganisms, so that the analytical techniques, in particular the multidetection according to the invention, may then be carried out.

This filtration means is based on the technique of ultrafiltration over hollow fibers in frontal mode.

The term “frontal mode”, as opposted to “tangential mode”, is intended to mean any one-pass passage of a starting liquid sample through the filtration means, with no recycling of at least part of the same sample at the inlet of said filtration means.

The use of this ultrafiltration means in frontal mode makes it possible to obtain concentrates having a small volume, and to perform the concentration of a sample within a timescale of the order of an hour at most, in a single passage, while at the same time guaranteeing the viability of the microorganisms, multirecovery, and yields of the order of 100%.

The term “multirecovery” is intended to mean the possibility of recovering, in the final sample, virtually all the various genera or species of microorganisms present in the starting sample.

These high yields are obtained on account of the inexistence of volumes, termed dead volumes, due for example, on other devices, to the presence of subsidiary piping, for example for recycling, and through the reliability of the porosity over the entire length of the hollow fiber.

The method of control according to the invention is thus applied to a sample optionally obtained by filtration, having a volume of between 1 ml and 100 liters.

A microorganism lysis step is carried out, either by mechanical lysis or chemical lysis, as described above.

A purification step is optionally applied, optionally using techniques of capture by oligonucleotides attached to magnetic particles, or using silica columns, silica particles (inert or magnetic), ion exchange columns, or any other method mentioned above.

An enzymatic amplification step is optionally applied, also preferentially using transcription techniques such as TMA, NASBA, but using in particular PCR and RT-PCR techniques.

An amplicon labeling step is applied, preferentially using a fluorescent label.

A hybridization step is then applied, preferentially using the specific identifying probes, or fragments thereof, attached to a solid support, and in particular using a biochip.

The method of control and the kit for microbiological determination according to the invention using these specific sequences make it possible to simultaneously detect a bacterium and/or a virus and/or a parasite from a set panel, in a single final multidetecton step.

Set panels of microorganisms can be easily defined as a function of the liquids to be analyzed.

Another subject of the invention is a method of production and/or disinfection of a liquid, characterized in that it comprises a step of analysis using a kit for microbiological determination as claimed in any one of claims 35 to 48 and generating an algorithm for interpretation of the data allowing said method of production and/or disinfection to be servocontrolled by said data generated by the kit for microbiological determination.

The advantages of the invention and the techniques used are illustrated by the nonlimiting examples and the attached figures.

### BRIEF DESCRIPTION OF THE DRAWINGS

FIG. 1 represents the evolution of the base-call on the probes specific for Escherichia coli and for Acinetobacter baumanii as a function of the number of copies of rRNA of each of the partners added before amplification. The boxed area in the graph represents the E. coli/A. baumanii proportions from which the E. coli targets are interpretable with the chip.

FIG. 2 represents the evolution of the base-call on the probes specific for E. coli, S. typhimurium and A. baumanii as a function of the proportions of the number of copies of labeled transcripts representing the 3 species.

### DETAILED DESCRIPTION OF EMBODIMENTS

In the examples described below, the strains used are:

Escherichia coli ATCC 11775

Enterococcus faecalis 19433T

Salmonella typhimurium API 9810059

Acinetobacter baumanii ATCC 19606

### EXAMPLE 1

### Detection and Identification of a Single Bacterial Cell in Culture: in the Case of Escherichia coli (gram −) and Enterococcus faecalis (gram +)

Preparation of the Culture

A strain of E. coli or E. faecalis is cultured at 37° C. in 2 ml of Luria Bertani broth. When the culture has reached an optical density at 620 nanometers of 0.2, 1 ml (10⁸ bacteria/ml) is removed. Serial dilutions are prepared, until 0.1 cell/μl is obtained.

Lysis of Micoorganisms

10 μl (1 cell) are removed from the suspension at 0.1 cells per microliter. 100 μl of a lysis buffer containing 10 mM Tris, 1 mM EDTA (dilution of a 100× TE solution marketed by SIGMA, ref. T-9285) and lyzozyme (Sigrna, ref. L-6876), the concentration of which is different depending on the Gram of the bacterium: 3 mg/ml for E. faecalis, 400 μg/ml for E. coli, are added to this suspension. The bacteria are lyzed by leaving the tube containing the bacterial suspension in contact with the lysis buffer for 5 to 10 min at ambient temperature.

Extraction and Purification of Nucleic Acids

This step is carried out using the Rneasy mini kit marketed by Qiagen (ref. 74104), according to the protocol recommended for the extraction and purification of bacterial total RNA.

RT-PCR

The two steps of RT and PCR will be carried out one after the other, in a single tube, using the ACCESS kit (ref A1250, Promega). For this, the 5× AMV/Tfl buffer, 1 mM of MgSO₄, 200 μM of dNTPs (deoxyribonucleoside triphosphates), 5 U of AMV RT polymerase, 5 U of Tfl polymerase, 5 U of RNAsin (Pranega ref. NZEIII), 0.5 μM of the eubacterial primers A1.1 and S9T7:

(SEQ ID NO:105)

5′ gaggcagcagtggggaat 3′

(SEQ ID NO:106)

5′ taatacgactcactatagggaggaggattactaccagggtatctaat
3′

(in bold: T7 polymerase promoter), are added to 25 μl of the total RNA suspension, in order to obtain 50 μl of final reaction volume.

For the RT step, the mixture is incubated for 45 min at 48° C., and then for 5 min at 94° C. For the PCR step, 35 cycles are then carried out, each composed of the following 3 steps: 94° C., 1 min; 55° C., 1 min; 68° C., 1 min. A final extension of 7 min at 68° C. is then carried out.

Verification of the Amplification

5 μl of amplification product (amplicon) are loaded onto a 1.5% agarose gel in EDTA Tris borate. After migration for 20 min at 200 V, the amplification band is visualized by staining with ethidium bromide and UV illumination. The amplification is shown to be positive by the presence of a band having the expected size (450 base pairs).

Identification of the amplicon on a DNA chip (Affymetrix, Santa Clara)

A biochip is synthesized on a solid support made of glass, according to the method described in U.S. Pat. No. 5,744,305 (Affymetrix, Fodor et al.) using the resequencing strategy described in application WO 95/11995 (Affymax, Chee et al.) and according to the method described by A Troesch et al. in J. Clin. Microbiol., vol. 37(1), p 49-55, 1999, with the following variants: the oligonucleotides synthesized on the chip perform the resequencing of the identifying sequences. This method makes it possible to decrease the total number of oligonucleotides synthesized and therefore has a considerable advantage in terms of production costs and without any compromise regarding the quality of the identification of the various microorganisms by virtue of the choice of these identifying sequences. The oligonucleotides comprise 20 bases, with an interrogation position at the 12th position relative to the 3′ end of the sequence. For the species E. coli and E. faecalis, there are also oligonucleotides of 17 bases, with 2 interrogation positions: one in the 10th position and one in the 8th position. Other oligonucleotides are between 10 and 25 nucleotides in length. The interrogation positions then vary as a function of the length of the oligonucleotide.

The analysis is performed on the complete GeneChip® system (reference 900228, Affymetrix, Santa Clara, Calif.), which comprises the GeneArray® reader, the GeneChip® hybridization oven, GeneChip® fluid station and GeneChip® analysis software.

1. Transcription and Labeling of Amplicons

Because of the antisense primer S9T7, all the amplification products have a T7 RNA polymerase promoter. These amplicons will then be used as matrix for a transcription reaction during which a fluorescent ribonucleotide will be incorporated. An aliquot (between 2 and 12 μl) is removed from the 50 μl of positive amplification product, and is added to a transcription mixture containing the components of the Megascript T7 kit from Ambion (ref. 1334), and fluorescein-12-UTP (Roche, ref. 1427857). The final reaction mixture is prepared in 20 μl and the transcription reaction takes place for 2 hours at 37° C.

2. Fragmentation of the Labeled Transcripts

In order to improve the hybridization conditions, the labeled transcripts are cleaved into fragments of approximately 20 nucleotides. For this, the 20 μl of labeled transcripts are subjected to the action of 30 mM imidazole (SIGMA) and 30 mM magnesium chloride (Merck) for 30 min at 65° C.

3. Hybridization on the Investigation Chip

A 5 μl aliquot is removed from the 20 μl of labeled and fragmented transcripts, and is added to 700 μl of hybridization buffer, the composition of which is 6× SSPE (Eurobio), 5 mM DTAB (Sigma), 0.5% Triton (Merck eurolab). This mixture is hybridized on the chip under the following conditions: 40 min at 45° C. After washing, the chip is scanned, and then the hybridization image obtained is analyzed using the Genechip© software (Affymetrix, Santa Clara). The hybridization spots make it possible to reconstitute the sequence of the amplicon, which is then compared with the reference sequences of the chip. The sequence (and therefore the species which corresponds to it) which exhibits the best percentage homogy (base-call, in %) with the sequence of the amplicon is selected for the identification.

4. Interpretation of the Results

Only part of the sequence of 450 bases is analyzed. It corresponds to all or part of the identifying probes represented on the biochip. The interpretation threshold, i.e. level of identification, is set at at least 70% of base-call on the identifying sequence. Below this threshold, the target is not identified.

Results

The RNA extracted from a single bacterial cell (E. coli or E. faecalis) gives rise to an amplification product, and then to a correct identification on the biochip.

### EXAMPLE 2

### Differentiation of Mixtures of 2 Different Bacterial Species

In this example, the eubacterial RT-PCR was applied to synthetic targets; that is to say these targets originate from the amplification, and then from the transcription of the 16S ribosomal DNA in its entirety. These targets are called in vitro transcripts. In this example, the target is a mixture of in vitro transcripts representing the species Escherichia coli and Acinetobacter baumanii. When the target is added to the RT-PCR tube, reasoning is no longer in terms of number of bacteria, but in terms of number of copies of in vitro transcripts, and then in number of bacteria equivalents, starting from the following premise: 1 bacterium corresponds to 104 copies of 16S ribosomal RNA.

For this, the transcripts were titered at 10¹¹ copies/μl. For Acinetobacter baumanii, a 10⁸copies/μl dilution is prepared. For Escherichia coli, dilutions of 10³/μl, 10⁴/μl, 10⁵/μl and 10⁶/μl are prepared. The conditions of the reaction mixture for the RT-PCR are identical to those described in Example 1, paragraph c), except that the target volume is no longer 25 μl of a total RNA suspension, but 2 μl of a mixture consisting of 1 μl of each dilution of transcript representing each species in the following proportions:

| | E. coli/A. baumanii bacteria equivalents | | | | | |
|---|---|---|---|---|---|---|
| | 0/0 | 0.1/10⁴ | 1/10⁴ | 10/10⁴ | 10²/10⁴ | 10⁴/0 |
| Copies of E. coli | 0 | 10³ | 10⁴ | 10⁵ | 10⁶ | 10⁸ |
| transcripts | | | | | | |
| Copies of A. baumanii | 0 | 10⁸ | 10⁸ | 10⁸ | 10⁸ | 0 |
| transcripts | | | | | | |

The sole amplicon obtained is then treated according to step e) of Example 1.

Results

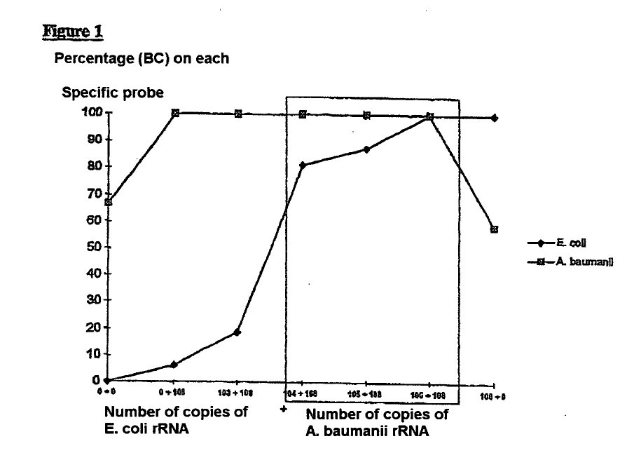

FIG. 1 shows that, by relating the number of copies of 16S rRNA back to a number of bacteria, it is therefore possible to detect, using the DNA chip, the equivalent of 1 E. coli in the presence of 10⁴ A baumanii, i.e. a proportion of 0.01%.

### EXAMPLE 3

### Differentiation of a Mixture of 3 Different Bacterial Species

Labeled transcripts of 3 bacterial species (Escherichia coli, Salmonella thyphimurium, Acinetobacter baumanii) are obtained according to protocole) at f.1. They are then purified using the Rneasy mini kit (Qiagen, ref. 74104) according to the protocol suitable for the purification of in vitro transcripts. The labeled transcripts are titered (reading at 260 nm on a spectrophotometer) so as to determine the number of targets (or copies) introduced into the hybridization mixture. The total number of copies in a hybridization mixture is set at 10¹³ copies.

The number of copies of the transcripts corresponding to the species E. coli is the same as that of the transcripts corresponding to the species S. thyphimurium. These transcripts were added with respect to the A. baumanii transcripts in the following way:

| | Proportion of E. coli-S. thyphimurium*/A. baumanii | | | | | |
|---|---|---|---|---|---|---|
| | 0.01% | 0.1% | 1% | 10% | 20% | 50% |
| Number of | 5 · 10⁸ | 5 · 10⁹ | 5 · 10¹⁰ | 5 · 10¹¹ | 10¹² | 2.5 · 10¹² |
| copies of E. | | | | | | |
| coli | | | | | | |
| transcripts | | | | | | |
| Number of | 5 · 10⁸ | 5 · 10⁹ | 5 · 10¹⁰ | 5 · 10¹¹ | 10¹² | 2.5 · 10¹² |
| copies of S. | | | | | | |
| thyphi- | | | | | | |
| murium | | | | | | |
| transcripts | | | | | | |
| Number of | 10¹³ | 10¹³ | 10¹³ | 10¹³ | 8 · 10¹² | 5 · 10¹² |
| copies A. | | | | | | |
| baumanii | | | | | | |
| transcripts | | | | | | |

Results

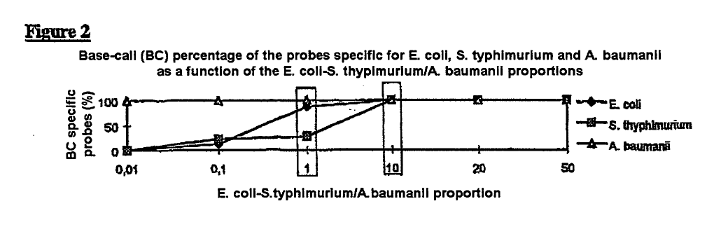

FIG. 2 shows that the detection of E. coli occurs at lower proportions (1%) than that of S. thyphimurium (10%). This result shows that it is possible to detect, on the chip, 3 different bacterial species.

### EXAMPLE 4

### Simultaneous Detection of Escherichia coli, Staphylococcus aureus and Salmonella enteritidis

a) Preparation of Bacterial Suspensions

Strains tested:

Escherichia coli ATCC 11775T

Staphylococcus aureus ATCC 12600T

Salmonella enteritidis ATCC 13076

The strains are cultured in a Trypticase Soy broth at 37° C. When the culture reaches an optical density of 0.2-0.3 (10⁸ bacteria/ml) 10-fold serial dilutions are prepared, until 100 bacteria/ml are obtained.

b) Mixing of Bacteria

The 3 bacterial species are mixed using the suspensions produced in section a), so as to have: 100 Escherichia coli, 100 Staphylococcus aureus and 100 Salmonella enteritidis.

c) Obtaining total RNA

1. Lysis of Microorganisms

The final volume will be 100 μl. 1 μl of 100× TE buffer (Sigma ref T-9285) is added, and lysozyme at 100 mg/ml (Sigma, Ref.L-6876) to have a final concentration of 10 mg/ml. The volume is then possibly made up with water (Sigma, ref. W-4502) to have 100 μl. Incubation is carried out for 30 min at 25° C.

2. Purification of Nucleic Acids

The RNeasy Mini Kit (Qiagen, ref 74104) is then used, applying the protocol recommended by Qiagen for bacteria.

d) RT-PCR

An RT-PCR is carried out using the ACCESS kit (Promega, ref. A1250) according to the protocol indicated in Example 1, section c).

e) Verification of the Amplification

According to the protocol indicated in Example 1, section d).

f) Analysis on a Biochip

1. Transcription and Labeling of the Amplicons

According to the protocol indicated in Example 1, section e)-1.

2. Fragmentation of the Labeled Transcripts

According to the protocol indicated in Example 1, section e)-2.

3. Hybridization on the Chip

According to the protocol indicated in Example 1, section e)-3.

4. Interpretation of the Results

The base-call on the identifying sequence corresponding to each of the taxons must be greater than 90%. Below this, the target is not identified.

Results

| | Base-call on the corresponding |
|---|---|
| Species tested | identifying sequence |
| Escherichia coli | 100% |
| Staphylococcus aureus | 100% |
| Salmonella enteritidis | 100% |

Conclusion

Simultaneous detection of the 3 bacterial species by hybridization on the corresponding identifying sequences was obtained.

### EXAMPLE 5

### Simultaneous Detection of Escherichia coli, Staphylococcus aureus, Salmonella enteritidis and Pseudomonas aeruginosa

a) Preparation of the Bacterial Suspensions

Strains Tested:
Escherichia coli ATCC 11775T
Staphylococcus aureus ATCC 12600T
Salmonella enteritidis ATCC 13076
Pseudomonas aeruginosa ATCC 10145T
   The bacterial suspensions are prepared according to the protocol indicated in Example 4, section a).

b) Mixing of the Bacteria

The 4 bacterial species are mixed using the suspensions produced in section a), so as to have: 100 Escherichia coli, 100 Staphylococcus aureus, 100 Salmonella enteritidis and 100 Pseudomonas aeruginosa.

c) Obtaining Total RNA

According to the protocols indicated in Example 4, section c).

d) RT-PCR

An RT-PCR is carried out using the ACCESS kit (Promega, ref. A1250) according to the protocol indicated in Example 1, section c).

e) Verification of the Amplification

According to the protocol indicated in Example 1, section d).

f) Analysis on a Biochip

1. Transcription and Labeling of the Amplicons

According to the protocol indicated in Example 1, section e)-1.

2. Fragmentation of the Labeled Transcripts

According to the protocol indicated in Example 1, section e)-2.

3. Hybridization on the Chip

According to the protocol indicated in Example 1, section e)-3.

4. Interpretation of the Results

The base-call on the identifying sequence of each of the taxons should be greater than 90%. Below this, the target is not identified.

Result

| | Base-call on the corresponding |
|---|---|
| Species tested | identifying sequence |
| Escherichia coli | 100% |
| Staphylococcus aureus | 91.9% |
| Salmonella enteritidis | 100% |
| Pseudomonas aeruginosa | 100% |

Conclusion

Simultaneous detection of the 4 bacterial species by hybridization on the corresponding identifying sequences was obtained.

### EXAMPLE 6

### Simultaneous detection of Escherichia coli, Cryptosporidium parvum and du Poliovirus Sabin 3

a) Preparation of the Suspensions

For Escherichia coli, the dilutions are prepared as indicated in Example 4, a). For Cryptosporidium parvum, serial dilutions are prepared from a suspension of oocystes with a titer of 10⁷/ml, marketed by Waterbome Inc. (St Louis, USA). For the Poliovirus Sabin 3, a suspension with a titer of 10⁹ PFU/ml is used.

b) Mixing of the Microorganisms

3000 E. coli, 3000 C. parvum and 3000 cfu of Poliovirus are mixed so as to obtain 300 μl of final volume.

c) Preparation of the Nucleic Acids

The 300 μl are prepared as 3×100 μl, since the extraction and the purification of the RNAs undergo 3 separate processes.

1. Escherichia coli

Preparation of the total RNA according to the protocol indicated in Example 4, section c)

2. Cryptosporidium parvum

The RNeasy Mini Kit (Qiagen, ref. 74104) is used according to a modified protocol. For this, added to the 100 μl are 350 μl of RLT lysis buffer from the RNeasy kit, and 25 μl of proteinase K at 19 mg/ml (Roche, ref. 1964372), which reduces to 1 mg/ml. This is left to act for 30 min at 65° C. The procedure is then continued according to the RNeasy Mini Kit protocol for bacteria.

3. For Poliovirus Sabin 3

40 μl of water (Sigma, ref. W-4502) are added to the 100 μl, and the Qiamp Viral RNA Mini Kit (Qiagen, ref. 52906) is used, according to the suppliers' instructions.

d) Amplification by RT-PCR

1. Escherichia coli

An RT-PCT is carried out using the ACCESS kit (Promega, ref. A1250) according to the protocol indicated in Example 1, section c).

2. Cryptosporidium parvum

An RT-PCT is carried out using the ACCESS kit (Promega, ref. A1250). For this, 25 μl of reaction mixture are added to the 25 μl of total RNA obtained in step c) 2, so as to have, in the final 50 μl: 1× AMV/Tfl buffer, 2.5 mM MgSO₄, 200 μM dNTPs, 5U of Tfl, 5U of AMV, 5U of RNAsin (Promega ref. N2111), and 200 μM primers XIA2F and XIA2R.

| | |
|---|---|
| XIA2F | |
| 5′ GGAAGGGTTGTATTATTAGATAAAG 3′ | (SEQ ID NO:107) |
| XIA2R-T7 | |
| 5′ taatacgactcactatagggaggaggatta | |
| AAGGAGTAAGGAACAACCTCCA 3′ | (SEQ ID NO:108) |

Taken from the publication by Xiao et al. in Applied and Environmental Microbiology, 1999 (in bold: T7 promoter of T7 polymerase, which will be used in the transcription).

For the RT step, the mixture is incubated for 45 min at 48° C. For the PCR step, incubation is carried out for 5 min at 94° C., and then 30 cycles are performed, each composed of the following 3 steps: 94° C., 45 sec; 55° C., 45 sec; 68° C., 1 min. A final extension of 7 min at 68° C. is then carried out.

3. Poliovirus Sabin 3

An RT-PCR is carried out using the ACCESS kit (Promega, ref. A1250). For this, 25 μl of reaction mixture are added to the 25 μl of total RNA obtained in step b) 2, so as to have, in the final 50 μl: 1× AMV/Tfl buffer, 2 mM MgSO₄, 300 μM dNTPs, 5U of Tfl, 5U of AMV, 5U of RNAsin (Promega ref. N2111), and 200 μM specific primers.

For the RT step, the mixture is incubated for 45 min at 48° C. For the PCR step, incubation is carried out for 2 min at 94° C., and then 40 cycles are performed, each composed of the following 3 steps: 94° C., 45 sec; 55° C., 30 sec; 68° C., 1 min. A final extension of 7 min at 68° C. is then carried out.

e) Verification of the Amplification

According to the protocol indicated in Example 1, section d).

f) Analysis on a Biochip

1. Transcription and Labeling of the Amplicons

According to the protocol indicated in Example 1, section e)-1.

2. Purification of the Labeled Transcripts

The 3 tubes containing the 20 μl of transcription are pooled and the purification is carried out using the RNeasy Mini Kit (Qiagen ref. 74104), protocol for the purification of in vitro transcripts. 20 μl of transcript are obtained.

3. Fragmentation of the Labeled and Purified Transcripts

According to the protocol indicated in Example 1, section e)-2.

4. Hybridization on the Chip

According to the protocol indicated in Example 1, section e)-3.

5. Interpretation of the Results

The base-call on the signature sequence of E. coli and C. parvum must be greater than 90%. For the Poliovirus 3, due to a sequence polymorphism, the detection threshold lies above 85%.

Result

| | Base-call on the corresponding |
|---|---|
| Species tested | identifying sequence |
| Escherichia coli | 100% |
| Cryptosporidium parvum | 100% |
| Poliovirus Sabin 3 | 88.9% |

Conclusion

Simultaneous detection of the 3 parameters by hybridization on the corresponding identifying sequences was obtained.

### EXAMPLE 7

### Simultaneous Detection of an Enterovirus (Coxsackievirus A9) and the hepatitis A Virus

1-Targets Considered:

Coxsackievirus strain A9 at 7 TCID₅₀/μL (extraction of nucleic acids using the Qiamp Viral RNA kit from Qiagen -ref. 52904—according to the suppliers' indications).

Vaccinal strain of the heptatitis A virus at 17.5 DICC₅₀/μL (extraction of nucleic acids using the Qiamp Viral RNA kit from Qiagen -ref. 52904—according to the suppliers' indications).

2-Multiplex RT-PCR

An RT-PCR is carried out using the ACCESS kit (Promega, ref. A1250). For this, 1 μL of each viral strain and 48 μL of the reaction medium are added such that in the following way:

| | Final concentration/tube |
|---|---|
| RNasin | 2.5U |
| 5X buffer | 1X |
| dNTP | 0.3 mM |
| Primers H1 + H2^{(a)} | 0.5 μM |
| Enterovirus primers^{(b)} | 0.3 μM |
| MgSO₄ | 2 mM |
| T4 gene 32 protein | 2.5 μg |
| AMV | 5U |
| Tfl | 5U |

^{(a)}publication Robertson et al., Virus Research, 1989, 13, 207-212
^{(b)}designated in the 5′NCR region

For the reverse transcription step, the mixture is incubated for 45 minutes at 48° C. After a denaturing step of 2 minutes at 94° C., the complementary DNAs obtained are amplified according to the following modalities: 45 cycles of [15 seconde at 94° C., 30 seconds at 55° C., 45 seconds at 68° C] with an elongation step of 7 minutes.

3-Verification of the Amplification

8 μL of RT-PCR products are loaded onto a 1.5% agarose gel in EDTA-Tris borate. After migration for 30 minutes under 100 V, amplification products are visualized by staining the gel with ethidium bromide and by UV illumination. Visualization of a band around 500 bp (enterovirus) and of another at 249 bp (HAV) shows that the amplification is effective.

Analysis on a Biochip

The amplification products are labeled according to patent WO99/65926.

Interpretation of the Results & Conclusions

The base-call on the sequence corresponding to each virus must be greater than 95%. Below this threshold, the target is not identified.

The following results are obtained:

| | % Base-Call |
|---|---|
| Coxsackievirus A9 | 96.7 |
| HAV | 96.9 |

Conclusion

Simultaneous detection of the 2 viral strains by hybridization on the corresponding identifying sequences was obtained.

1. (canceled)

2. A substrate having oligonucleotide probes immobilized on the substrate, the oligonucleotide probes respectively comprising the sequences set forth in SEQ ID NOs: 5, 6, 9-11, 15, 40-49, 53-55, 59, 60, 62-66, 68-75 and 97, and/or the full-length complements of the sequences set forth in SEQ ID NOs: 5, 6, 9-11, 15, 40-49, 53-55, 59, 60, 62-66, 68-75 and 97.

3. A reagent comprising a mixture of oligonucleotide probes, the oligonucleotide probes of the mixture respectively comprising the sequences set forth in SEQ ID NOs: 5, 6, 9-11, 15, 40-49, 53-55, 59, 60, 62-66, 68-75 and 97, and/or the full-length complements of the sequences set forth in SEQ ID NOs: 5, 6, 9-11, 15, 40-49, 53-55, 59, 60, 62-66, 68-75 and 97.

4. A method comprising:
a) obtaining a sample comprising nucleic acid;
b) contacting the sample comprising nucleic acid with the substrate according to claim 2; and
c) detecting formation or non-formation of a hybridization complex between the nucleic acid and at least one of the oligonucleotide probes.

5. The method according to claim 4, wherein the oligonucleotide probes are DNA and/or RNA.

6. The method according to claim 4, wherein the nucleic acid of the sample comprising nucleic acid is at least one member selected from the group consisting of DNA, RNA, mRNA and rRNA.

7. The method according to claim 4, further comprising prior to step b), subjecting the sample to one or more treatment steps selected from the group consisting of an enriching step, a lysis step, a purification step, an amplification step and an amplicon labeling step.

8. The method according to claim 4, further comprising prior to step b), carrying out at least one step of enriching the sample comprising nucleic acid.

9. The method according to claim 8, further comprising carrying out the enriching step by filtration.

10. The method according to claim 9, further comprising carrying out the filtration by ultrafiltration of the sample over hollow fibers in frontal mode.

11. The method according to claim 10, further comprising subsequent to the filtration step, carrying out at least one lysis step.

12. The method according to claim 11, further comprising subsequent to the lysis step, carrying out at least one purification step.

13. The method according to claim 12, further comprising subsequent to the purification step, carrying out at least one amplification step.

14. The method according to claim 13, further comprising carrying out the amplification step via PCR, RT-PCR, LCR, 3SR, NASBA, or TMA.

15. The method according to claim 13, further comprising subsequent to the amplification step, carrying out an amplicon labeling step.

16. The method according to claim 4, further comprising labeling the nucleic acid in the sample containing nucleic acid and/or the oligonucleotide probes.

17. The method according to claim 4, further comprising carrying out step b) by performing hybridization, PCR, in vivo excystation, FISH, DOT-BLOT, Southern blot, Northern blot, sandwich assay, or a combination thereof.

18. The method according to claim 4, further comprising completing the method in a time of less than 24 hours.

19. The method according to claim 4, wherein the sample comprising nucleic acid comprises nucleic acid from one or more microorganisms, said microorganisms being independently selected from bacteria, parasites and viruses, wherein:
the bacteria are selected from the group consisting of species from the E. coli genus, species from the Salmonella genus, Pseudomonas aeroginosa, and species from the Legionella genus;
the parasites are selected from the group consisting of species from the Cryptosporidium genus and Giardia lamblia; and
the viruses are selected from the group consisting of enteroviruses, Hepatitits A Virus and Norwalk virus.

20. The method according to claim 19, wherein the bacteria is E. coli serotype 0157:H17.

21. The method according to claim 19, wherein the bacteria is Legionella pneumophila.

22. The method according to claim 19, wherein the parasite is Cryptosporidium parvum.

23. The method according to claim 4, wherein the method is used for identification, quantification and/or determination of viability of microorganisms.

24. The method according to claim 4, wherein the method distinguishes among three groups of parasites, the three groups of parasites consisting of dead parasites, viable and infectious parasites, and viable and non-infectious parasites.
